(19) United States
(12) Patent Application Publication
(10) Pub. No.: US 2022/0118206 A1
(43) Pub. Date: Apr. 21, 2022

(54) AN AIRWAY MANAGEMENT DEVICE AND METHODS OF MANUFACTURING AN OBJECT

(71) Applicant: Ronald Craig WIGHT, Singapore (SG)

(72) Inventor: Ronald Craig WIGHT, Singapore (SG)

(21) Appl. No.: 17/428,792

(22) PCT Filed: Feb. 5, 2020
(86) PCT No.: PCT/SG2020/050053

§ 371 Date: Aug. 5, 2021

Related U.S. Application Data
(60) Provisional application No. 62/803,122, filed on Feb. 8, 2019.

(51) Int. Cl. A61M 16/04

(52) U.S. Cl. CPC A61M 16/0488; A61M 16/0463; A61M 16/0459; A61M 16/0409; A61M 2207/10; A61M 16/0447

(57) ABSTRACT

An airway management device has a body (6) including an external shell moulded from a polypropylene copolymer (PP) blended with a thermoplastic elastomer (TPE) of styrene-ethylene/butylene-styrene (SEBS), the external shell extending from a proximal opening to a distal tip of the body (6), the external shell having a curved portion (35) and a linear portion (37). Methods of manufacturing are also disclosed.

This application claims the benefit of U.S. Provisional Patent Application Ser. No. 62/803,122, the disclosure of which is incorporated herein by reference. All publications, patents and patent applications mentioned in this specification are herein incorporated by reference to the same extent as if each individual publication or patent application was specifically and individually indicated to be incorporated by reference.

### BACKGROUND

The laryngeal mask airway (LMA) has become an alternative to tracheal intubation or the face mask for the management of the airway during general anaesthesia. The LMA “Classic” (CLMA) is an artificial airway device manufactured from liquid silicone rubber (LSR), comprising a curved or flexible tube opening at one end into the interior of an oval shaped, hollow and inflatable mask portion, whose fit and function occupy the space behind the larynx and seal around the circumference of the laryngeal inlet. The device does not penetrate the interior of the larynx, hence the avoidance of vocal cord trauma. A meta-analysis of 858 publications (Brimacombe, “The advantages of the LMA over the tracheal tube or facemask: a meta-analysis,” Can J Anaesth 1995; 42: 1017-23), specific to the CLMA determined that the CLMA offered increased speed and ease of placement, haemodynamic stability and improved oxygen saturation compared to the endotracheal tube (ETT) and the facemask (FM). The single biggest disadvantage compared to the ETT being the higher frequency of gastric insufflation and for the FM, gastro-oesophageal reflux (GOR). Overall, it was noted that the insertion technique may have been inadequately defined with the possibility that, suboptimal positioning may have influenced the results. It was also noted that the CLMA causes an increased work of breathing (WOB) compared with the ETT.

A later study (Roux M, Drolet P, Girard M, Grenier Y, Petit B, “Effect of the laryngeal mask airway on oesophageal pH: influence of the volume and pressure inside the cuff,” Br J Anaesth. 1999 April; 82(4):566-9) confirmed the higher incidence of GOR when using the CLMA compared with using a face mask and an oropharyngeal airway, by comparing the pH levels in the oesophagus during anaesthesia. Lower average pH in the lower oesophagus, as well as an increased percentage time where the pH was below 4.0 was evident when using the CLMA. There was no established correlation between cuff pressure or inflation volume and the incidence of GOR.

A further study (Reissmann H, Pothmann W, Fullekrug B, Dietz R, Schulte am Esch J., “Resistance of laryngeal mask airway and tracheal tube in mechanically ventilated patient,” BJA: British Journal of Anaesthesia, Volume 85, Issue 3, 1 Sep. 2000, Pages 410-416) compared the inspiratory airflow resistance of the CLMA and ETT in mechanically ventilated patients. Although airflow resistance for a size 4 LMA should be less than an appropriately sized ETT (8.5 mm internal diameter), the anatomical structures between the LMA sealing against the laryngeal opening and the trachea are of such variability, that the mean airflow resistance of the CLMA and larynx together, offered no clinically relevant difference compared to an ETT. Furthermore, it was concluded that presence of the CLMA in the hypopharynx might actually change upper airway geometry causing a narrowing of the glottic opening, further contributing to airflow resistance.

The intubating laryngeal mask airway (ILMA) was disclosed by Brain in U.S. Patent No. U.S. Pat. No. 6,079,409A. The flexible airway tube of the CLMA was replaced with an anatomically curved wide bore stainless steel tube equipped with a proximal guiding handle. Brain et al. concluded that the ILMA appeared on initial assessment to be an effective ventilatory device and intubation guide for routine and difficult airway patients not at risk of gastric aspiration (Brain A I J, Verghese C, Addy E V, Kapila A, Brimacombe J., “The intubating laryngeal mask. II: A preliminary clinical report of a new means of intubating the trachea,” BJA: British Journal of Anaesthesia, Volume 79, Issue 6, 1 Dec. 1997, Pages 704-709). Compared to the standard CLMA, the ILMA is configured with a larger diameter but shorter airway tube sufficiently rigid so as to guide the appropriate length of ETT through the mask and into the glottis. Tracheal intubation with the ILMA was successful in 149 out of 150 patients (99.3%) with 75 (50%) of these patients intubated at the first attempt. The ILMA required significantly fewer adjusting manoeuvers in patients with a potential or known airway difficulty. The ETT used for the study was a prototype characterised by a straight cuff and a flexible silicone tube.

Positioned in the hypopharynx and creating a barrier at the upper oesophageal sphincter (OES), the primary limitation of the CLMA and its variants is that a patient's lungs are not reliably protected from regurgitated stomach content (Keller C, Brimacombe J, Bittersohl J, Lirk P, von Goedecke A., “Aspiration and the laryngeal mask airway: three cases and a review of the literature,” BJA: British Journal of Anaesthesia, Volume 93, Issue 4, 1 Oct. 2004, Pages 579-582). Keller et al evaluated three cases of aspiration with the LMA where bile stained fluid was removed from the trachea. In each case the LMA was replaced with an ETT. In one of these cases, an ILMA was used but aspiration occurred before intubation could be performed. Hence, prior assessment of aspiration risk was deemed critical to determining if an LMA should be used and if so, which type of LMA. The ILMA has recorded increased oropharyngeal leak pressure compared to the CLMA but the pharyngeal mucosal pressures are higher and exceeded capillary perfusion pressure. Therefore, the ILMA is not suitable as a routine airway management device and should be removed once intubation is completed (Keller C, Brimacombe J., “Pharyngeal Mucosal Pressures, Airway Sealing Pressures, and Fiberoptic Position with the Intubating versus the Standard Laryngeal Mask Airway,” Anesthesiology 4 1999, Vol. 90, 1001-1006). Supraglottic Airway Devices, where supraglottic means “above the larynx” have been categorized as either first generation or second generation (Cook T, Howes B., “Supraglottic airway devices: recent advances,” Continuing Education in Anaesthesia Critical Care & Pain, Volume 11, Issue 2, 1 Apr. 2011, Pages 56-61). Hence, the CLMA and the ILMA are referred to as first generation SAD as neither offer protection against gastric aspiration.

The LMA “Proseal” (PLMA), as described in U.S. Patent Application Publication No. 2012/0211010A1, is a second generation SAD because it is configured with a built in conduit for gastric drainage (GD) whereby the alimentary and respiratory tracts are separated permitting access to or escape of stomach fluid, reducing the risk of gastric insufflation and pulmonary aspiration (Brain A I J, Verghese C, Strube P J., “The LMA ‘ProSeal’—a laryngeal mask with an oesophageal vent,” British Journal of Anaesthesia, Volume 84, Issue 5, May 2000, Pages 650-654). It is accepted that in the resting state, the hypopharynx is usually closed however, any SAD occupying the hypopharynx sufficiently to form an oesophageal seal and provide for GD, must open the oesophagus and in so doing, push the glottis anteriorly (O'Neil M J., “Mechanical closure of the vocal cords with the LMA ProSeal,” Br J Anaesth 2002; Volume 89, Issue 6, Pages 936-937). Brimacombe et al reported vocal cord closure associated with a reduction in the anteroposterior diameter of the glottic inlet (Brimacombe J, Richardson C, Keller C, Donald S., “Mechanical closure of the vocal cords with Proseal laryngeal mask airway,” Br J Anaesth 2002; 89:296-7) when using the PLMA in fully paralysed patients. It was postulated that the mechanism of vocal cord closure was caused by the inflatable cuff, compressing the glottic inlet along the anteroposterior axis, thereby reducing the tension in the vocal cords and allowing the arytenoid cartilages to rotate inwards and the vocal cords to close. Although over-inflation of the cuff was not specifically stated, withdrawing air from the cuff and moving the patient's head to the sniffing position reduced the compressive force against the glottis, allowing the arytenoids to rotate outwards and the vocal cords to open. Brimacombe noted 4 out of 915 (0.4%) paralysed patients managed with the PLMA exhibited mechanical cord closure and a degree of epiglottic down folding was seen in 17% of patients but it rarely caused airway obstruction due to the accessory vent under the drain tube. In another reported case, (Ghai A, Hooda S, Wadhera R, Kad N, Garg N., “Failed ventilation with LMA Proseal in a patient with sleep apnea syndrome,” Anaesth Pain & Intensive Care 2013; 17(1):94-96), the investigators removed and replaced the PLMA with an alternative device after several attempts to overcome failed ventilation. The authors cited Stacy et al (Stacy M R, Sivasankar R, Bahlmann U B, Hughes R C, Hall J E., “Mechanical closure of the vocal cords with the airway management device,” Br J Anaesth 2003; 91:299) reporting a similar 20% incidence of airway obstruction with SAD. Stacy et al hypothesized the epiglottis was down folding or mechanical cord closure. Insertion of the PLMA beyond its optimal position results in near complete airway obstruction, presumably because of forward displacement of the glottic inlet.

The LMA “Supreme” (SLMA), as disclosed in U.S. Patent Application Publication No. 2012/0145160 A1, is also second-generation SAD characterised by an inflatable cuff and oesophageal gastric drain tube (GD), but is a single use device manufactured from semi-rigid PVC and vinyl elastomer. A case study by Bergmann et al evaluated two methods of cuff inflation and device fixation of the SLMA to determine the effect on oropharyngeal leak pressure, position of the distal tip in the hypopharynx, separation of gastrointestinal and respiratory tracts and perioperative airway morbidity (Bergmann I, Crozier T A, Roessler M, Schotola H, Mansur A, Buttner B, Hinz J M, Bauer M., “The effect of changing the sequence of cuff inflation and device fixation with the LMA-Supreme® on device position, ventilatory complications, and airway morbidity: a clinical and fiberscopic study,” BMC Anesthesiology. 2014; 14: 2. Published online 2014 Jan. 4. doi: 10.1186/1471-2253-14-2). The control method was the manufacturer recommended method of sequential insertion, cuff inflation and device fixation. The alternative “study” method fixed the device prior to cuff inflation. No discernible differences concerning the incidence of inadequate ventilation or incorrect positioning, oropharyngeal leak pressure, tip position and gastrointestinal and respiratory tract were observed. Importantly, glottic narrowing also occurred with equal frequency. However, glottic narrowing accompanied by impaired ventilation occurred in the control group significantly more than the study group. Sore throat, hoarseness, blood on the SLMA and dysphagia was significantly higher with the control group. A further study (Lopez A M, Valero R, Hurtado P, Gambus P, Pons M, Anglada T., “Comparison of the LMA Supreme with the LMA Proseal for airway management in patients anaesthetized in prone position,” British Journal of Anaesthesia 107 (2): 265-71 (2011)), comparing PLMA with SLMA concluded that although both devices were similar in rate of first time insertion, PLMA required fewer manipulations and exhibited slightly higher oropharyngeal leak pressure. The incidence of laryngospasm was similar in both devices and successfully treated by increasing the depth of anaesthesia and giving a neuromuscular blocking agent if required. Laryngospasm (Gavel G, Walker R W M, “Laryngospasm in anaesthesia,” Continuing Education in Anaesthesia Critical Care & Pain, Volume 14, Issue 2, 1 Apr. 2014, Pages 47-51), has been described as sustained closure of the vocal cords resulting in the partial or complete loss of a patient's airway. It is a primitive reflex protecting against aspiration that can be problematic under general anaesthesia. Therefore, it can be concluded that the pathogenesis of glottic narrowing or vocal cord closure, can either be mechanical as well as physiological, the latter occurring as a result of innervation during insertion of the SAD, or if anaesthesia is light.

I-gel is another second generation SAD that features a non-inflatable cuff and the possibility to introduce a gastric catheter (Theiler L, Gutzmann M, Kleine-Brueggeney M, Urwyler N, Kaempfen B, Grief R., “I-gel™ supraglottic airway in clinical practice: a prospective observational multicentre study,” British Journal of Anaesthesia 109 (6): 990-5 (2012)), to drain away gastric fluid. It relies on the soft SEBS (Styrene-Ethylene/Butylene-Styrene) gel like mass of the cuff to conform to the anatomical variances of the laryngeal inlet. The authors noted the difficulties arising during insertion due to the bulk of the non-inflatable cuff i.e. it could not be deflated to a flat profile to ease passage past the teeth and tongue. Insertion and subsequent fixation cause the tongue to protrude outwards and to be clenched between the teeth and the proximal end of the relatively straight airway tube.

A study of oesophageal seal efficacy for the I-gel compared to the PLMA and SLMA in cadavers showed that when the distal cuff of the PLMA and the SLMA is correctly placed in the hypopharynx, the respective sealing pressure is three and two times that of the I-gel (Schmidbauer W, Bercker S, Volk T, Bogusch G, Mager G, Kerner T., “Oesophageal seal of the novel supralaryngeal airway device I-Gel™ in comparison with the laryngeal mask airways Classic™ and ProSeal™ using a cadaver model,” BJA: British Journal of Anaesthesia, Volume 102, Issue 1, 1 Jan. 2009, Pages 135-139). Cadavers are not accurately representative of the human body, nonetheless, the study reinforced that SAD do not prevent aspiration as reliably as the ETT. A magnetic resonance imaging study of the in vivo position of the I-gel compared to the SLMA by Russo et al showed that both devices had a significant impact on the glottis, more so the SLMA due to the larger inflatable cuff (Russo S G, Cremer S, Eich C, Jipp M, Cohnen J, Strack M, Quintel M, Mohr A., “Magnetic resonance imaging study of the in vivo position of the extraglottic airway devices i-gel™ and LMA-Supreme™ in anaesthetized human volunteers,” Br J Anaesth. 2012 December; 109(6):996-1004). The SLMA protruded deeper into the UOS, the I-gel causing a greater dilation at the upper level of the UOS. The anatomical airway of the SLMA had little effect on the lingual soft tissue whereas the straighter and semi-rigid I-gel compresses the tongue contributing to higher mucosal pressure. Whilst it is possible to intubate through the I-gel, the relatively straight airway lacks anatomical curvature reducing the rate of first-time insertion when compared to the ILMA.

In addition to mechanical closure of the vocal cords, Lingual (Brimacombe J, Clarke G, Keller C. Lingual nerve injury associated with the ProSeal laryngeal mask airway: a case report and review of literature. Br J Anaesth 2005; 95:420-3), hypoglossal and recurrent Laryngeal (Michalek P, Donaldson W, Votrubova E, Hakl M. Complications Associated with the Use of Supraglottic Airway Devices in Perioperative Medicine. Biomed Res Int. 2015. Article ID 746560, 13 pages) nerve injuries have been reported in various case studies regardless of inflatable cuff or no-inflatable cuff. In relation to Lingual, the most probable cause of such injury being pressure neuropraxia from the airway tube (lingual) or cuff (hypoglossal and recurrent laryngeal). Two contributing factors were cited. The selected PLMA was undersize and therefore cuff size too small, and secondly, the use of nitrous oxide. Over inflation of the undersized cuff to improve efficacy of the seal and the gradual increase in intra-cuff nitrous oxide diffusion (Cros A M, Pitti R, Conil C, Giraud D, Verhulst J. Severe Dysphonia after Use of a Laryngeal Mask. Anesthesiology 1997; 86:497-500), particularly if the procedure is prolonged, increase the cuff pressure. Concerning Laryngeal nerve injury, Michalek et al concluded the aetiology of neurological injury is multifactorial with the cuff being the significant contributing factor, either too rigid during insertion or direct compression of nervous structures whilst in situ.

The CLMA offered increased speed and ease of placement, haemodynamic stability and improved oxygen saturation over the ETT. However, it cannot reliably protect the lungs from regurgitated stomach content. Variations of the CLMA included the ILMA configured with a larger diameter and shorter airway tube, sufficiently rigid so as to guide a flexible ETT through the mask and into the glottis. Tracheal intubation with the ILMA has been successful but provides no access to or escape of stomach content to reduce the risk of pulmonary aspiration. Furthermore, it is not recommended to be used as a routine airway device.

Rate of first-time insertion, over inflation of the cuff and incorrect positioning are frequently cited in the literature. When describing the relationship between cuff pressure and volume, Bick et al (Bick E, Bailes I, Patel A, Brain Al. Fewer sore throats and a better seal: why routine manometry for laryngeal mask airways must become the standard of care. Anaesthesia. 2014 December; 69(12):1304-8), established an inflatable cuff goes from negative elastic recoil at low volumes to positive recoil at higher volumes. The recommended inflation volume for a size 4 CLMA, without distorting or distending the LSR cuff material, is 30 ml. The pharynx, though not as rigid as the trachea, does significantly oppose distention. Once inserted, the inference is that inflation volume to maintain a satisfactory oropharyngeal leak pressure is actually less than 30 ml, hence the evidence linking sore throats with excessive cuff pressure. Increased mucosal pressure and failure to conform to the contours of the larynx, pharynx and oesophagus are also the direct consequence of over inflation. Presence of an LMA in the hypopharynx changes upper airway geometry causing a narrowing of the glottic opening, further contributing to airflow resistance and an increased WOB compared with the ETT.

It can be summarised that the characteristic bulky construction of second-generation SAD manufactured from Liquid Silicone Rubber such as the PLMA or PVC elastomers such as the SLMA, factor significantly in the pathogenesis of vocal cord closure and the causation or aetiology of neurological injury. Concerning the PLMA, U.S. Patent Application Publication No. 2012/0211010 A1 (page 1, para 005) teaches the drain tube conduit must be sufficiently rigid at its distal end to withstand the pressure of the inflated cuff and it has been found that this may make proper insertion of the deflated device into the patient's throat more difficult that either necessary or desirable.

U.S. Patent Application Publication No. 2012/0211010 A1 discloses a reinforced backplate 27 [page 9, para 0111] that has been thickened in relation to first generation SAD. Included, is an inflatable volume described as the back-cuff 65 (FIG. 7 and FIG. 8) which is created by a flexible panel 62 [page 4, para 0051], draped over the backplate 27 and adhesive bonded to the posterior of the main cuff 40 along a perimeter 63. The main cuff 40 and the back-cuff 65 are interconnected so as to inflate simultaneously. When inflated, pressure within the back-cuff 65 bears against the oval portion of the backplate 27 causing it to herniate anteriorly and potentially displacing the internal drain tube 115 anteriorly. To ameliorate this condition, this reference teaches that the backplate has to be thickened and moulded using a higher durometer hardness Liquid Silicone Rubber (LSR) material than the backplate of a first-generation SAD. To offset the additional bulk of this configuration, the flexible panel 62 is moulded as a thin sheet of LSR capable of considerable elongation in response to the inflation pressure within.

Functionally [page 9, para 0108], inflation of the main cuff 40 causes expansion of the distal region 45 enabling it to lie against and adapt to the pharynx 197 and hypopharynx 212. Upon further inflation, the back-cuff 65 causes initial engagement between the flexible panel 62 and posterior surface of the pharynx 197. The pressure within the back-cuff 65 urges the main cuff 40 anteriorly, pressing against the tissue surrounding the laryngeal inlet 67. This tightens the sealing engagement between the main cuff 40 and the tissue surrounding the laryngeal inlet 67, thereby reducing leakage between such tissue and the main cuff 40. An initial description of this configuration in the installed and inflated condition is described in page 4, para 0051. Specifically, an increased anterior-posterior space characterized as a minimum depth of 10 mm [page 9, para 0109], measured between the anterior tangency of the internal drain-tube 115 and the plane described by the anterior surface of the main cuff 40 (FIG. 9 “b”). To maintain the ideal anterior-posterior dimension, the distal orifice 123 must be wedged into the upper esophageal sphincter so that when the main cuff 40 is inflated, the internal drain-tube 115 is surrounded by an annular inflated volume.

Furthermore, U.S. Patent Application Publication No. 2012/0211010 A1 teaches that over-inflation [page 9, para 0109] of the back-cuff 65, will cause the oval portion 87 of the backplate 27 to bulge anteriorly resulting in displacement of the internal drain tube 115 relative to the main cuff 40 and loss of the aforementioned anterior posterior space. If this anterior-posterior space decreases below a minimal level, the internal drain tube may impinge against the anatomical structures of the throat 32 normally present in the laryngeal chamber region 110. Hence, the reported cases of vocal cord closure associated with a reduction in the anteroposterior diameter of the glottic inlet when using the PLMA.

Similarly, with the SLMA of U.S. Patent Application Publication No. 2012/0145160 A1 (page 1, para 0010) the provision of a gastric discharge opening at the distal end of the mask applicable for direct service of the hypopharynx has resulted in a tendency for such masks to become bulky and unduly stiff, thus making for difficulty in properly inserting the mask. Specifically, (page 2, para 0013) in any such device regardless of the material from which it is formed, adding an oesophageal drain in itself adds greatly to complexity of manufacture and can also affect the performance of devices, in terms of ease of insertion, seal formation and prevention of insufflation.

This complexity can be exacerbated still further by using PVC or similar performing viscous polymers. Viscous polymers possess a viscosity factor or time dependent strain rate. When a load is applied, then removed, the release of energy is not immediate but time dependent. During insertion and subsequent manipulation of the SAD by various methods not limited to rotation and tilting, this characteristic viscosity factor contributes to back-folding and over-folding of the main cuff or distal portion of the cuff, as described by Anaesthetic and respiratory equipment—Supralaryngeal airways and connectors, ISO 11712:2009(E). The result of such folding is incorrect seal formation and the high likelihood of insufflation.

Furthermore, the need to provide a drain tube that is sealed from the airway and passes through the inflatable cuff poses a particularly difficult problem. In terms of effects on functionality, provision of a drain tube can cause unacceptable stiffening of the mask tip area and occlusion and or restriction of the airway passage. The relatively rigid PVC airway tube 2 of the SLMA (page 3, para 0049) includes grooves or channels 20, configured either side 18 and 19 of the airway tube 2 (FIG. 1, FIG. 3 and FIG. 10) to improve resilience during insertion and to prevent kinking. The oesophageal drain tube 41 is inserted into the airway tube 2 (page 4, para 0062) and secured by adhesive to the connector body 43 at the proximal end and the backplate 4 at the distal end. This provides for fluid communication in the minor bore 49, separate from fluid communication in the major bore 48 i.e. the interior of the airway tube 2. The addition of ribs and channels increases the bulk of the assembled device 1, the external grooves and channels (20) forming ridges along the inner surfaces of the sides of the airway tube reducing the interior space of the airway tube 2. The oesophageal drain tube 41 occupies the median plane of this interior space. Aligned and bonded with adhesive to the connector body 43 and plug 45, the configuration of the oesophageal drain tube 41 effectively hinders intubation.

Alternative SADs with a non-inflatable cuff have shown less glottic displacement with a correspondingly lower oesophageal seal pressure. In this instance, the bulky airway does not offer anatomical curvature and compresses the tongue causing it to protrude outwards with subsequent higher mucosal pressure. Whilst intubation is possible, the maximum size of ETT is less than the recommended for the same weight e.g. a size 4 I-gel accommodates an ID 7.0 mm ETT whereas the recommended ETT ID for the same body mass is 8.0 mm to 8.5 mm.

Typically, the oesophageal drain tube must pass through the inflatable cuff and in doing so poses a particularly difficult manufacturing problem. In addition, provision of the drain tube creates unacceptable stiffening of the distal tip affecting performance, in terms of ease of insertion, seal formation and prevention of insufflation. Semi-rigid PVC and PVC elastomers commonly used for a single use disposable SAD exacerbate the aforementioned difficulties because acceptable flexural performance requires increased thickness, which in turn creates bulk.

Notwithstanding, there is a need for a single use SAD that can combine gastric drainage and tracheal intubation of an appropriately sized ETT, via an anatomical curved airway and which can also be used as a routine airway management device; and contrary to past approaches, the bulk of the device can be significantly reduced by not exposing the oesophageal drain tube (alternatively referred to as gastric drain tube) to the inflation pressure within the cuff.

WO2015119577 described an airway management device comprising a body having a proximal end for receiving an oxygen supply tube and a distal end. To reduce the bulk of the distal end, the bulk of the entire device, in proportion, must be reduced. This precludes use of adhesives and redefines the method of manufacture and selection of materials. Past approaches have proposed passages or conduits for fluid communication as separate or independent components. There has been no attempt to combine these passages so that their wall thicknesses and features can be shared to reduce overall bulk. Except where the cuff portion of the SAD has been limited to a gel like mass unable to be deflated for ease of insertion (I-gel), the rheological relationship between a polyolefin such as polypropylene (PP) and block co-polymer SEBS (Styrene-Ethylene/Butylene-Styrene) has not been fully exploited. SEBS is a thermoplastic elastomer (TPE), characterized by hard and soft domains within individual polymer strands. The end-blocks of these strands are crystalline styrene while the mid-blocs are soft ethylene-butylene blocks. The strands join at the styrene end-blocks forming a physical cross-link that provides a rubber like elasticity.

Accordingly, a need is identified for an improved airway management device that overcomes some or all of the above-identified limitations, or others yet to be discovered.

### SUMMARY

According to one aspect of the disclosure, an airway management device is provided. In one embodiment, the device comprises a body including an external shell moulded from a polypropylene copolymer (PP) blended with a thermoplastic elastomer (TPE) of styrene-ethylene/butylene-styrene (SEBS), the external shell extending from a proximal opening to a distal tip of the body, the external shell having a curved portion and a linear portion.

In some embodiments, the device further includes an intermediate strip moulded from a polypropylene copolymer (PP). The intermediate strip may be attached to the external shell intermediate to the curved portion and the linear portion.

In some embodiments, the device may further include a first over-mould of SEBS comprising a posterior contour and a distal contour on the external shell. The first over-mould may comprise a distal perimeter defining a first opposed edge of an over-moulded cuff membrane continuing tangentially from said perimeter as a toroidal curve whose end point is in spaced relation and normal to the first opposed edge, the end points defining an open posterior perimeter or second opposed edge and a linear portion over-moulding a proximal end such that said curved portion and the linear portion are joined as a single moulding by planar sealing voids to first and second sides of an intermediate strip. A second over-mould of SEBS may close the open length of membrane, forming an inflatable cuff.

In some embodiments, the posterior contour of the body is adapted to be located within a hypopharynx, and a distal end is adapted to be located within an upper oesophageal sphincter creating an oesophageal seal, immediately superior to a distal opening. The anterior compound curvature of the external shell is an internal posterior surface of a passage or gastric drain tube reducing the bulk of the distal tip. The device may further include a surrounding contour over-moulding the anterior compound curvature of the external shell and which is adapted for locating and pressing against the hypopharynx, the distal to proximal full length configuration of the external shell providing resistance against displacement of the distal opening superiorly from increasing oesophageal pressure. The drain tube and a drain tube distal opening may be integral with a distal posterior contour not surrounded by an annular volume of the inflatable cuff.

A closed tubular section of the device may form a chamber providing a space for a distal portion of the inflatable cuff with a posterior displacement when inflated. In some embodiments, through any horizontal cross section of the inflatable cuff, first and second edges are provided for at least the length of the distal portion of the gastric drain tube. The edges may be generally or approximately parallel to a median plane of the curved portion, such that the width between the first and second edges after second over-moulding is equal to an outer diameter of the distal drain tube. A curvature of the inflatable cuff membrane between the first and second edges is a single contiguous curve of uniform durometer hardness, sealing posteriorly against a hypopharynx and anteriorly against a laryngeal inlet without adhesive joint.

The disclosure also pertains to a method of using an airway management device. The method may comprise providing a removable connector/adaptor on the linear portion to reduce a length from a proximal opening of the body through to a trachea, thereby providing additional depth of insertion of a distal tip of an endotracheal tube. Also disclosed is a method of using an airway management device, comprising providing a finger stopper creating a fixed position to rest a thumb during insertion, to grip a proximal end when removing the device after intubation and to act as a depth indicator, with reference to teeth, when the device is in situ.

This disclosure also pertains to a method of forming an airway management device. The method comprises providing a body including polypropylene copolymer (PP) and thermoplastic elastomer (TPE) of styrene-ethylene/butylene-styrene (SEBS). The body includes an external shell moulded from a majority PP copolymer blended with SEBS extending from a proximal opening to a distal tip of the body.

The method may further include the step of attaching an intermediate strip moulded from PP copolymer to said external shell intermediate to a curved portion and a linear portion of the body. Still further, the method may include providing a first over-mould of SEBS comprising an initial posterior contour and distal contour over-moulded onto the external shell, whose distal perimeter defines a first opposed edge of an over-moulded cuff membrane that continues tangentially from said distal perimeter as a toroidal curve whose end point is in spaced relation and normal to the first opposed edge, the end points collectively defining an open posterior perimeter or second opposed edge and a linear portion over-moulding the proximal end such that said curved portion and the linear portion are joined as a single moulding by planar sealing voids to lateral sides of the intermediate strip. The method may further include the step of providing a second over-mould of SEBS closing said membrane, forming an inflatable cuff and completing the body.

Independent or dependent on the foregoing, the disclosure also pertains to a method of forming an object, which may be applied to an airway management device, but could have broader applicability as well. The method comprises injection moulding a first portion of the object over a first core associated with a fixture. After the injection moulding of the first portion, the method comprises moving a second core associated with the fixture to a deployed position. The method further comprises injection moulding a second portion of the object over the second core and the first portion.

In some embodiments, the step of moving the second core associated with the fixture to the deployed position comprises rotating the second core relative to the fixture. In some embodiments, the method includes attaching a pre-formed part to the object. The method further includes placing one or more removable cores into the object, and placing one or more removable cores inside the injection mould prior to moulding a second portion of the object. The method also includes over-moulding a membrane as part of the second portion of the object.

The method may further include the step of injection moulding a third portion closing and sealing the membrane to form an inflatable portion of the object. The method may further include removing the removable cores from the membrane of the object prior to injection moulding the third portion.

In some embodiments, the step of injection moulding the first portion is completed in a first mould including the fixture. The step of injection moulding the second portion may be completed in a second mould including the fixture. The step of injection moulding the third portion to close and seal the membrane may be completed in a third mould including the fixture.

The method may further include the step of transferring the fixture from a first mould to a second mould between the steps of injection moulding the first portion and second portion of the object. The step of injection moulding the first portion of the object over the first core associated with the fixture comprises forming the external shell of the object. The method may further include the steps of moving the first core to release a proximal end of the object, and removing the object from the second core of the fixture. The method may include providing a body comprising polypropylene copolymer (PP) and thermoplastic elastomer (TPE) of styrene-ethylene/butylene-styrene (SEBS), characterized in that the first portion comprises an external shell moulded during the first injection moulding step from a majority PP copolymer blended with SEBS extending from a proximal opening to a distal tip of the body. Any of these methods may be applied to manufacture or form an airway management device.

Still a further aspect of the disclosure pertains to an apparatus for forming an injection moulded object. The apparatus comprises a reconfigurable fixture including a first movable core over which a first portion of the injection moulded object is formed and a second movable core over which a second portion of the injection moulded object is formed. In some embodiments, the first movable core is adapted for rotating relative to the fixture, and the second movable core may also be adapted for rotating relative to the fixture.

In some embodiments, a first removable core is adapted for being removably attached to the fixture. The first removable core may comprise a connector for connecting to the fixture, as well as a handle. The fixture may comprise a retainer, such a spring, for maintaining the second movable core in a deployed position.

Yet a further aspect of the disclosure pertains to a method of manufacturing an airway management device. The method comprises providing a tubular body having a linear portion and a curved portion, the tubular body including a plurality of supports adjacent to a posterior channel. The method further comprises providing an intermediate strip in engagement with the plurality of supports and overlying the posterior channel. The method also includes over-moulding material onto the intermediate strip.

In some embodiments, the method comprises the steps of: (1) injection moulding a first portion of the body of the airway management device over a first core associated with a fixture; (2) after the injection moulding of the first portion, moving a second core associated with the fixture to a deployed position; and (3) injection moulding a second portion of the body over the second core and the first portion of the body. The step of moving the second core associated with the fixture to the deployed position may comprise rotating the second core relative to the fixture.

In some embodiments, the method further includes the steps of placing one or more removable cores in the tubular body, placing one or more removable cores inside the second injection mould, and over-moulding the removable cores with a membrane as part of the second portion of the body. The method may further include removing said removable cores from close proximity to the first core and second core leaving an open membrane, and over-moulding the second portion with a third portion closing and sealing the open membrane to form an inflatable cuff on the tubular body. The method may further include the step of moving the first movable core and removing the removable cores to release the tubular body.

In some embodiments, the method may include placing a first material in one or more voids adjacent the intermediate strip with a first material. The method may further include the step of melting a portion of the intermediate strip comprising a second material so as to diffuse the first material into the second material.

A further aspect of the disclosure pertains to a method of forming an object. The method comprises, in a first injection mould, injection moulding a first portion of the object over a first core associated with a fixture. The method further comprises placing the fixture in a second injection mould, and injection over-moulding a second portion of the object over the second core associated with the fixture and partially or wholly over the first portion. Furthermore, placing the fixture in a third injection mould and injection over-moulding a third portion over the first and second portion.

In some embodiments, the first core is movable relative to the fixture, and further including the step of moving the first core following the injection moulding of the first portion or the second portion of the object. The step of injection moulding the second portion of the object may comprise injection moulding over a second core associated with the fixture. The second core may be movable relative to the fixture, and the method may further include the step of moving the second core to a deployed position after the step of injection moulding the first portion of the object and prior to the injection moulding of the second portion of the object.

In some embodiments, the method may further include the step of placing one or more removable cores inside the second injection mould prior to the step of injection moulding the second portion of the object. The step of injection moulding the second portion of the object may comprise over-moulding an open membrane onto the one or more removable cores. The one or more removable cores may be removed from the second injection mould together with the fixture. Furthermore, the one or more removable cores may be removed from the open membrane after injection moulding the second portion and prior to injection moulding the third portion of the object. The method may further include the step of closing and sealing the open membrane to form an inflatable portion of the object.

The disclosed apparatus and methods may be used to form any object, including without limitation to the present disclosure, an airway management device having any size, shape, or form.

### BRIEF DESCRIPTION OF DRAWINGS

FIG. 1 is an isometric view of a body of an airway management device according to one embodiment;

FIG. 2 is an isometric view of an insert for the body of FIG. 1;

FIG. 3 is an isometric view of the body of FIG. 1;

FIG. 4 is an isometric view of the insert of FIG. 2;

FIG. 5 is an isometric view of an oxygen supply adaptor of an airway management device according to another embodiment;

FIG. 6 is a cross sectional view of the body of FIG. 7;

FIG. 6a is a detail cross sectional view of the body of FIG. 7;

FIG. 7 is an isometric view of the body of FIG. 1;

FIG. 8 is an isometric view of the body of FIG. 1;

FIG. 9 is an elevation view of the body of FIG. 1;

FIG. 10 is a plan view of the body of FIG. 1;

FIG. 11 is a front view of a body of an airway management device according to a further embodiment;

FIG. 12 is a back view of the body of FIG. 11;

FIG. 13 is a detail cross sectional view of the body of FIG. 12;

FIG. 14 is a detail cross sectional view of the body of FIG. 12;

FIG. 15 is an isometric and cross-sectional view of the body of FIG. 12;

FIG. 16 is an isometric view of the body of FIG. 12;

FIG. 17 is an isometric of a receiving tube of an airway management device;

FIG. 18 is a back of a body of an airway management device;

FIG. 19 is a back of a body of an airway management device;

FIG. 20 is a side elevation view of the body of FIG. 18;

FIG. 21 is a side elevation view of the body of FIG. 19;

FIG. 22 is a back-elevation view of the body of FIG. 18;

FIG. 23 is a back-elevation view of the body of FIG. 19;

FIG. 24 is an isometric view of a body of an airway management device;

FIG. 25 is an isometric view of the body of FIG. 24;

FIG. 26 is a front view of a body of an airway management device according to a further embodiment;

FIG. 27 is a partially cut-away front view of the embodiment of FIG. 26;

FIG. 28 is a partially cross-sectional, enlarged end view of the embodiment of FIG. 26;

FIG. 29 is a cross-sectional side-view of the embodiment of FIG. 26;

FIG. 30 is presents cross-sectional views of the embodiment of FIG. 26 under different operating conditions;

FIG. 31 is a side view of a further embodiment;

FIG. 32 is an isometric view of the FIG. 31 embodiment; and

FIGS. 33 and 34 are views of an airway management device in situ.

FIG. 35-45 relate to a method of forming an object, such as the airway management device(s) of FIGS. 1-34, as one example, using an injection moulding technique.

### DETAILED DESCRIPTION

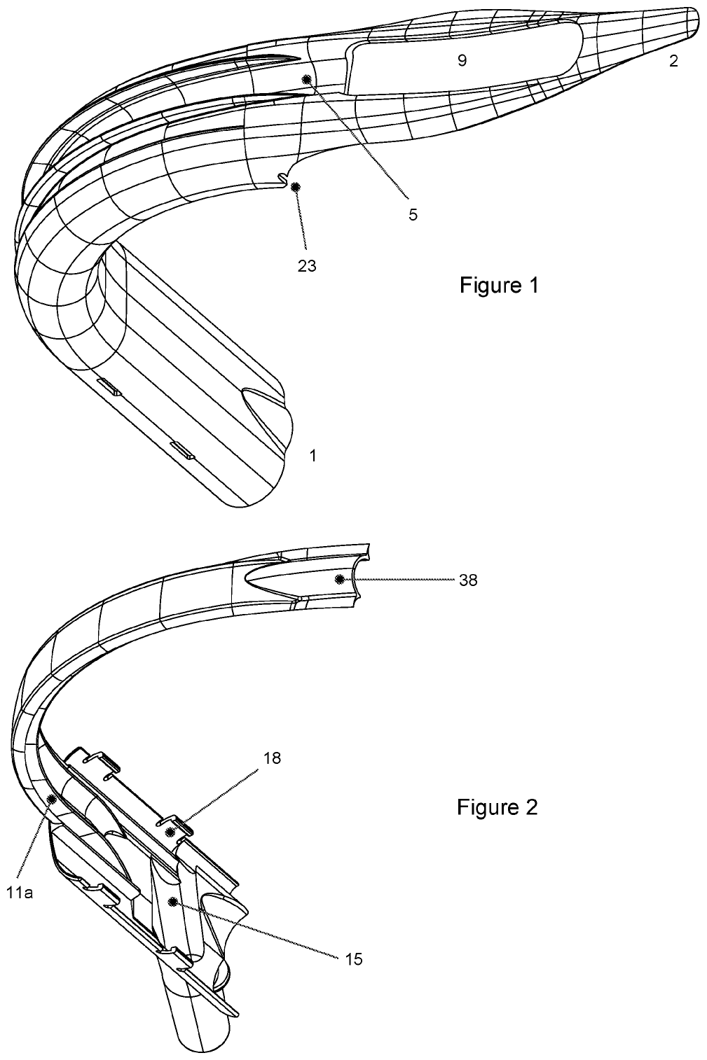

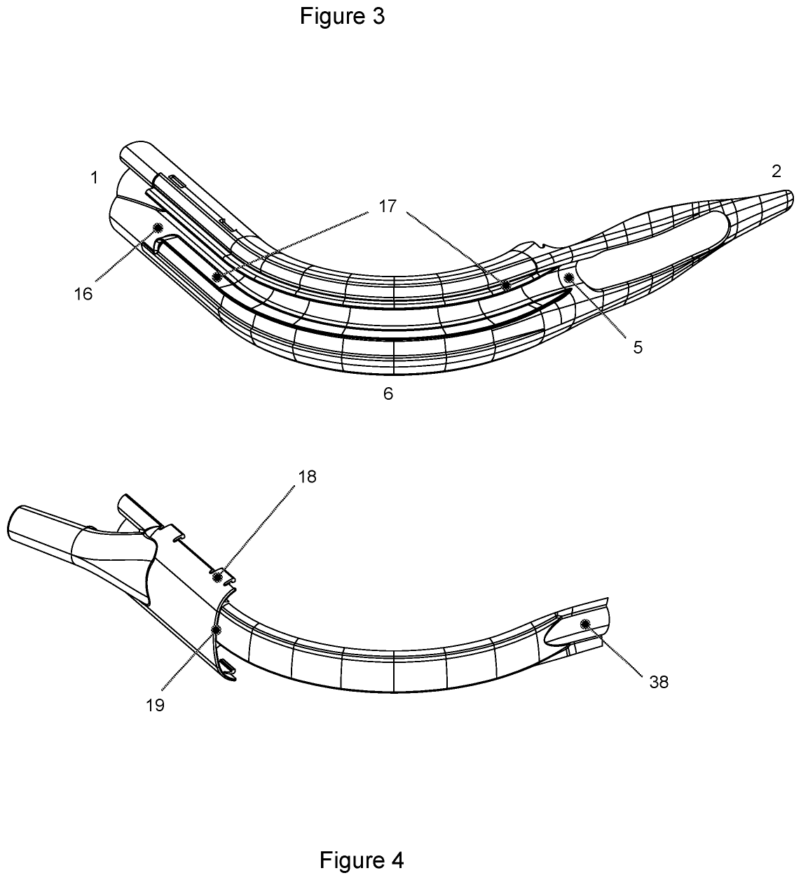

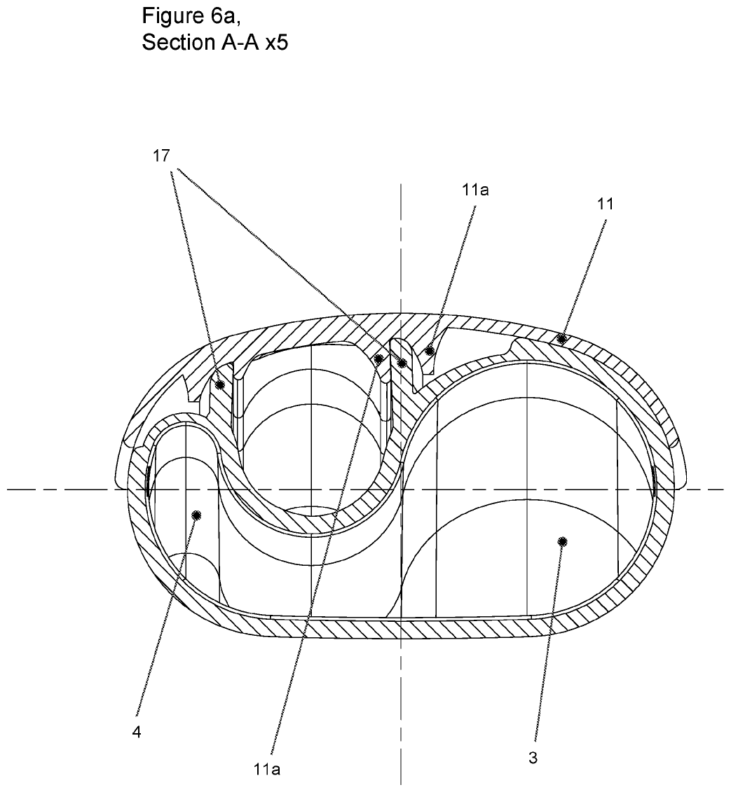

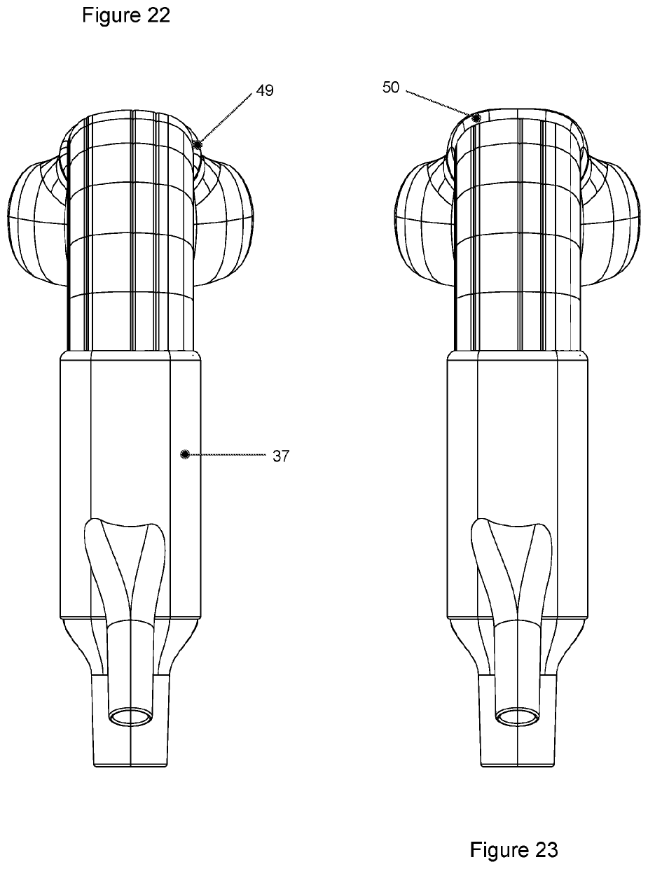

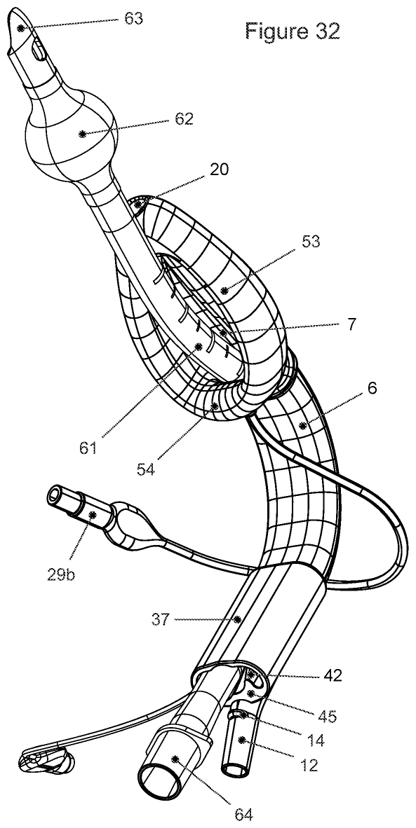

With reference now to FIGS. 1-34, various embodiments of an airway management device are disclosed. The airway management device includes a body 6, such as the airway tube (FIGS. 1 and 3) extending from the proximal end 1 of the device through to the distal tip 2. The horizontal cross section A-A (FIG. 6) through the straight portion of the proximal airway tube, shows the primary 3 and secondary passage 4 configured either side of the median plane. This configuration forms a shell providing a first moment of area greater than a similarly dimensioned circular or elliptical cross section. This provides the device with sufficient flexural strength and so acting as an exoskeleton as compared with prior art devices where much of the flexural strength is derived from components within the device, and so demonstrating an endo-skeleton structure.

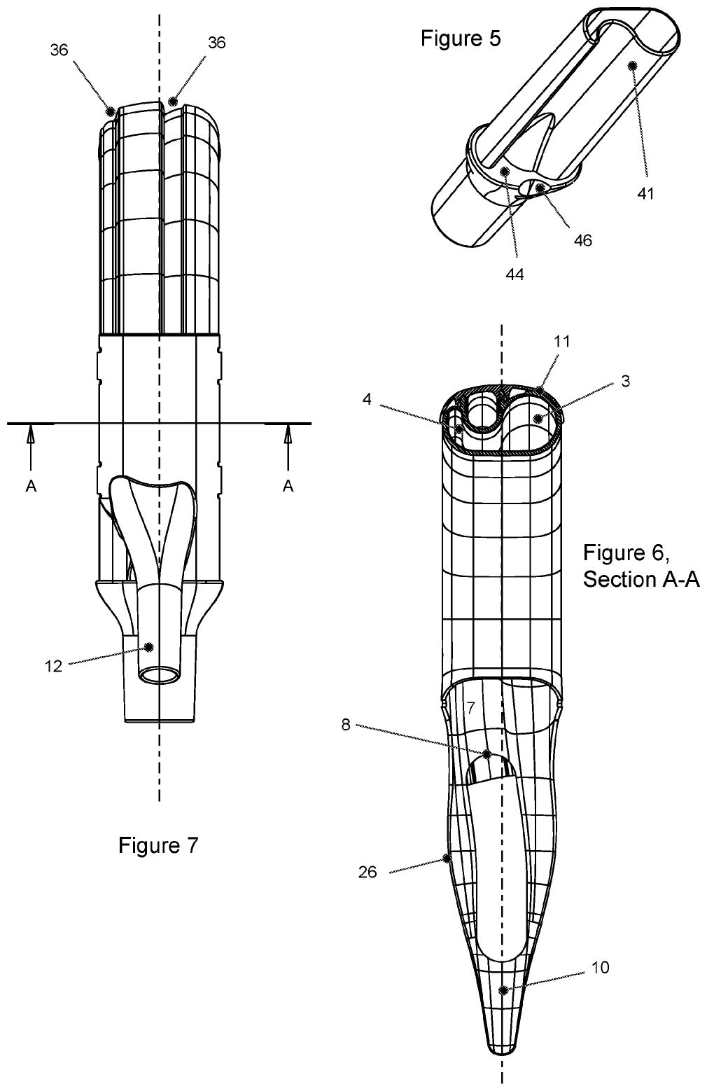

Inserted into the airway tube proximal opening is an adaptor (FIGS. 5 and 17), which facilitates connection to an oxygen supply as well as combining into a more rigid structure able to cope with and to facilitate the forces of circumduction during insertion. The parallel and sagittal planar relationship of these two passages defines an additional partial posterior channel 5 that, together with an intermediate strip (FIGS. 2 and 4), creates a laterally offset third passage to facilitate gastric drainage.

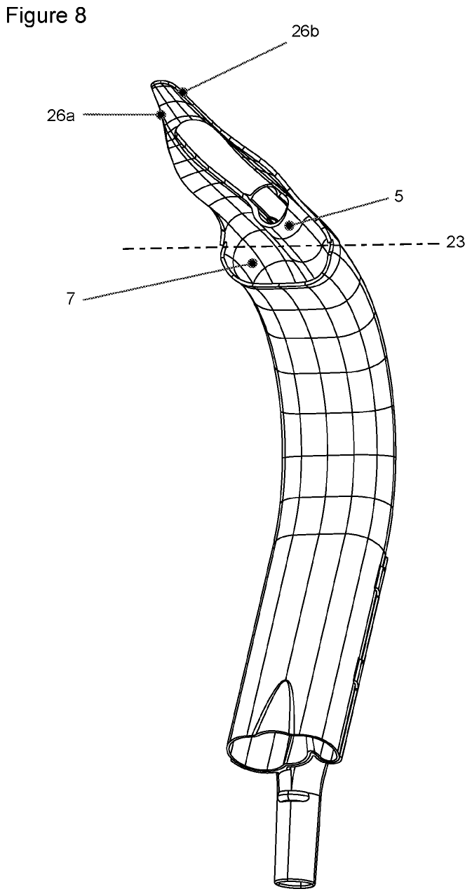

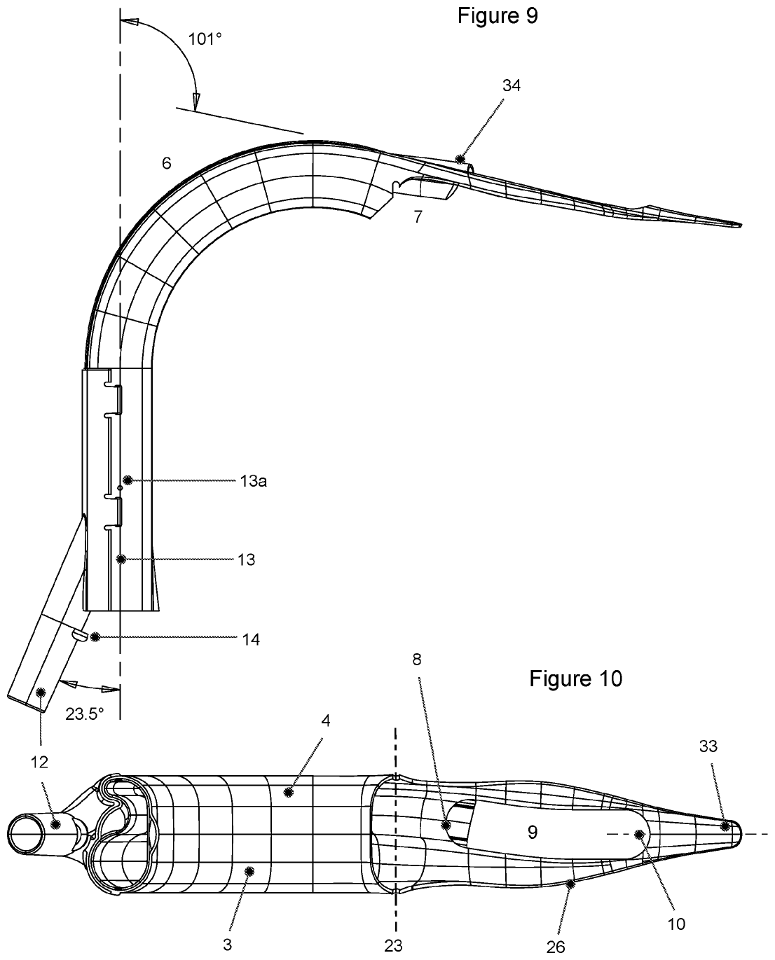

Cross section A-A of FIG. 6 progresses inferiorly through an anatomically approximated curvature of approximately 101 degrees (FIG. 9), parallel to the median plane, whereupon it transitions from a closed cross section to an open cross section (FIGS. 8 and 9) coinciding with the ventral opening of the device 7, where the primary and secondary passages terminate openly. Within this opening, the primary passage provides gaseous communication. When the adaptor is removed, this primary passage 3 allows for blind intubation (FIG. 10). The secondary passage 4 provides for endoscopic access during blind intubation as well as a secondary passage for spontaneous breathing during blind intubation.

Continuing inferiorly from this transition, the airway tube cross section maintains the semi-circular contour of the partial posterior channel 5 until reaching the proximal end 8 of the medial slot 9, a feature congruent with the anterior or ventral opening. When viewed anteriorly toward the frontal plane (FIGS. 6 and 10), the medial slot provides a route of progressive curvature for gastric drainage, from the partial posterior channel 5 through the medial slot to the anterior side of the distal airway tube; aligning the route for gastric drainage to the median plane of the distal tip 10; allowing the passing of a gastric drainage or suction tube with minimal frictional resistance.

Attached to the posterior of the airway tube is the intermediate strip (FIGS. 2 and 4) which exhibits curvature in the sagittal plane matching the airway tube and horizontal cross section 11 providing geometric conformance and attachment to the airway tube (FIGS. 6 and 6a). The proximal intermediate strip (FIGS. 9 and 10) is defined by a tubular feature 12 that serves as the entry point for gastric drainage or suction tube; and whose median axis when viewed laterally, adopts an angle of approximately 23.5 degrees with the horizontal plane coincident with the median axis 13 through the proximal end of the airway tube (FIGS. 9 and 21). When positioned on the airway tube, the intermediate strip covers the partial posterior channel 5 which is essentially an elongate recess which together defines a third passage as a route for gastric drainage. The intermediate strip, once positioned, is flush with an external surface of the body of the device. The distal end of the intermediate strip terminates at the proximal end of the medial slot 8. Continuing inferiorly to the distal extremity 2, the airway tube cross section progressively reduces in width and first moment of area. Horizontal cross sections throughout this transition exhibit ventrally concave curvature i.e. maintaining the posterior contour 34 were the intermediate strip (FIGS. 2, 4 and 9) to continue until the distal extremity of the airway tube.

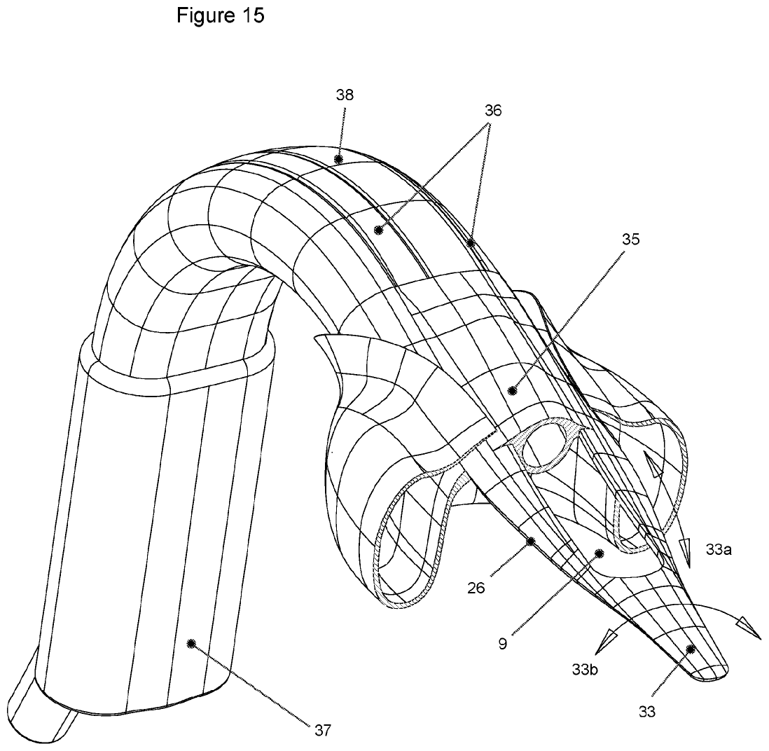

When combined with the elastic properties of the polyolefin material, the ventrally concave curvature parallel to the medial slot 33a and horizontally through 33b the medial slot creates a compound curvature (FIG. 15) or partial conical spring (Belleville washer) that encourages an immediate elastic response from the polyolefin material during flexure; thus maintaining contact between posterior airway tube and the posterior hypo-pharynx during insertion, without excessive force contributing to co-morbidity and soft tissue damage.

In pure mechanical terms, the distal end of the airway tube can be considered as the fixed support, whilst the airway tube by itself can be considered to act as a cantilever beam. Force exerted through the straight proximal portion of the airway tube during insertion concentrates flexion and extension through a horizontal axis coincident with two laterally opposed slots 23. The primary passage being larger in diameter than the secondary passage allows a degree of rotation around the medial axis of the proximal airway tube that can be transferred as torsion through to the distal tip. SAD's using semi-rigid PVC materials for the airway tube behave in a viscous manner i.e. when force is applied, they resist shear and exhibit linear strain (relationship change in length to original length) for the duration of the applied force. However, these forces are dissipated into the PVC material such that when force is released, PVC will not immediately respond and return to its original state. This lost energy, or hysteresis, is a significant disadvantage of prior art based on PVC materials. Polyolefin materials such as polypropylene exhibit a superior viscoelastic response, characterised by elastic rather than viscous response.

During insertion, the forces transferred through the airway tube are manifested by circumduction. Consequently, hysteresis in the materials used by existing prior may prevent the distal tip being correctly in situ with the upper oesophageal sphincter. Prior art describes the possibility of the distal tip entering the larynx or, the distal tip of the LMA or SAD may fold under, a phenomenon described as down-folding. Unlike other LMA or SAD, this invention uses an airway tube that extends from the proximal end to the distal tip and whose form and function utilise the more immediate viscoelastic response of a rigid polyolefin material. Where other SAD's describe a ventral displacement of the distal tip in relation to a dorsal or posterior reference point on the airway tube to better conform to the anatomy, this invention provides for a wide range of flexional response that obviates the ventral displacement described by prior art.

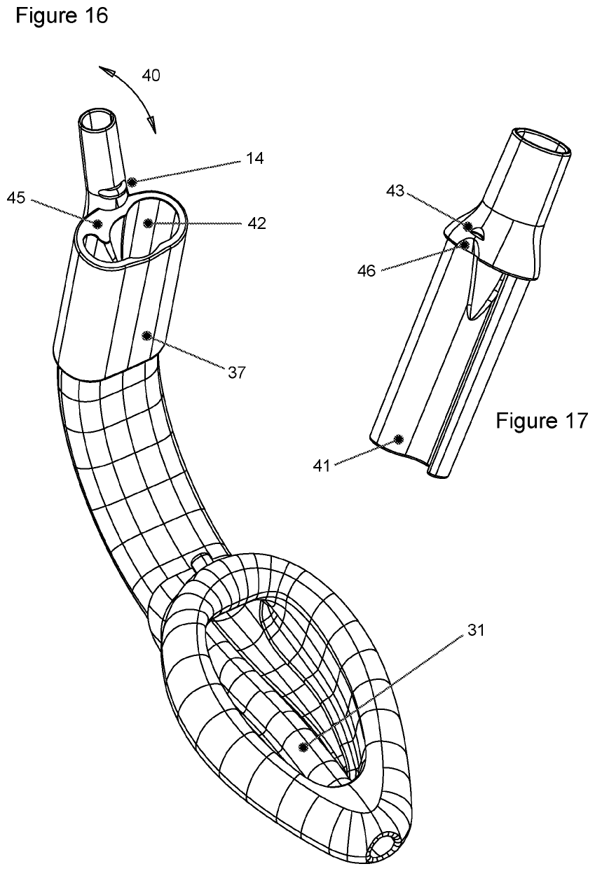

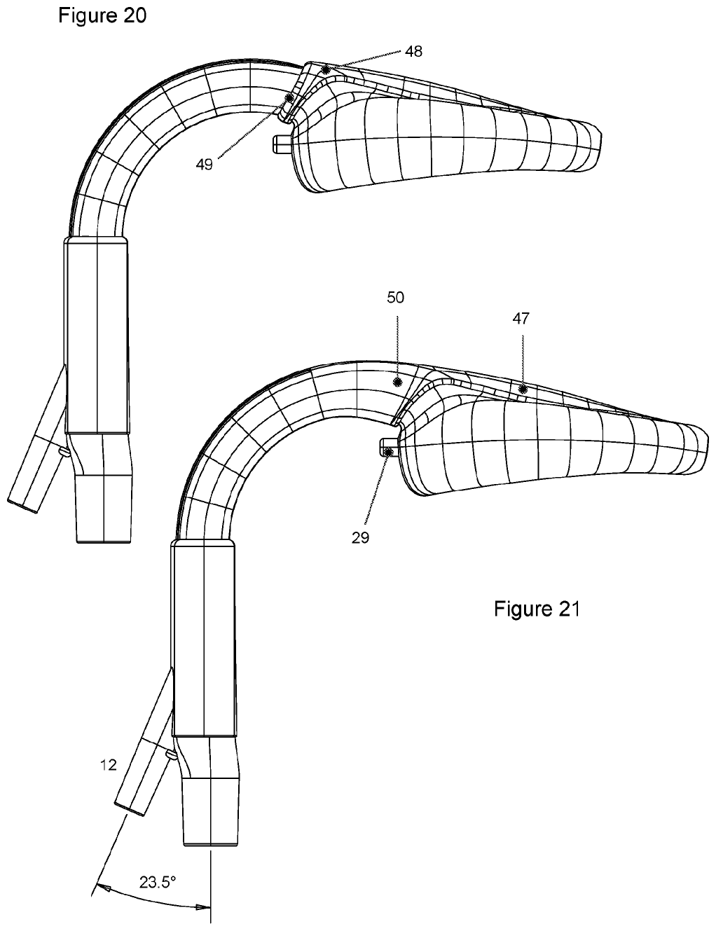

Protruding from the external surface of the gastric drain tube opening in closest proximity to the adaptor (FIG. 16), a raised step 14 is defined that has a corresponding cut out (FIG. 17) or notch 43 in the outer surface of the adaptor. This raised step retains and prevents the adaptor from separating away from the airway tube (FIG. 20).

When viewed superiorly toward the distal tip (FIG. 7), the proximal end 12 of the gastric drain tube is aligned with the median plane of the airway tube i.e. both passages share common a mid-plane (FIG. 7). It must be noted that the mid-plane of the airway tube is with reference to the lateral extremities of the airway tube rather than an alignment with the primary or secondary passage. To provide a primary passage of sufficient internal diameter to accommodate the insertion of an ETT and blind intubation, the third passage is laterally offset and divided by an impermeable barrier (posterior surface of airway tube) to ensure simultaneous blind intubation and gastric access.

As the median axis of the proximal opening (FIG. 9) approaches an intersection with the median axis of the adaptor and airway tube 13a, the tubular cross section transitions increasingly elliptical and no longer exhibits an enclosed perimeter, having opened up 15 to straddle the proximal airway tube (FIG. 2). When a gastric drainage suction tube is inserted through the tubular feature 12, the distal tip of the suction tube will make tangential contact 16 with posterior surface of the primary passage (FIG. 3). Further insertion deflects the suction tube laterally, seeking alignment with the supporting structure of supports, such as ribs 17 adjacent to the posterior channel or third passage in this region. The suction tube can then be guided inferiorly to exit at the distal tip of the device 20.

Proximally, the intermediate strip is attached by 4 latches, 2 per side positioned laterally 18 where the intermediate strip straddles the airway tube. Coinciding at the tangent where the straddling straight section of the airway tube terminates and the curvature 6 begins, the intermediate strip narrows abruptly 19. The supporting structure of ribs 17 follow the curvature of the airway tube 6; opposing ribs 11a integral with the intermediate strip (FIG. 6a) provide alignment and minimal interference, sufficient to provide for aforementioned attachment. Ribs 11a and 17 progressively diminish and terminate at the proximal end of the medial slot 8.

Having described the airway tube, intermediate strip and adaptor, any or all of which may be manufactured from polyolefin material in one possible embodiment, the description now focuses on the inflatable cuff manufactured from a thermoplastic elastomer (TPE) compounded from the same base polyolefin material. This in itself provides the means of assembly for the device described herein. The self-adhering property of TPE, adheres the intermediate strip to the airway tube and create an open thin walled cuff membrane by virtue of an initial injection moulding processes; a subsequent injection moulding process entraps the open membrane and creates an airtight and inflatable cuff, integral to the form and function of the device.

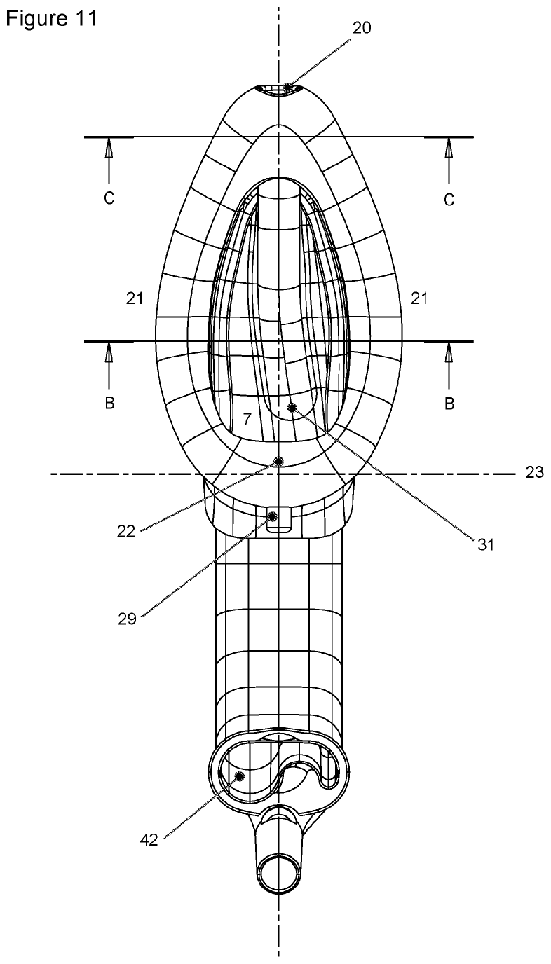

Viewed anteriorly toward the frontal plane (FIG. 11), the initial injection moulding process surrounds the perimeter of the distal airway tube with an elliptical shape cuff membrane of TPE, in a generally toroidal shape about the airway tube. In a specific embodiment, the cuff membrane may be characterised by; a distal tip whose curvature and width facilitate the tubular distal opening of the third passage 20 or gastric drain tube; lateral extremities 21 defined by curvature extending superiorly and tangential to the distal tip; an increasing rate of change of curvature that closes the elliptical shape at the median plane 22, just superior to the horizontal axis through two laterally opposed slots 23 and; an enclosed third passage or gastric drain tube 24, totally covering the medial slot 9 and whose contour and curvature 31 reconcile with that of the partial posterior channel 5. It will be appreciated that in alternative embodiments, the membrane may be a variety of open shapes, which may allow closure through a second moulding process to seal the open membrane and thus permit inflation of the cuff.

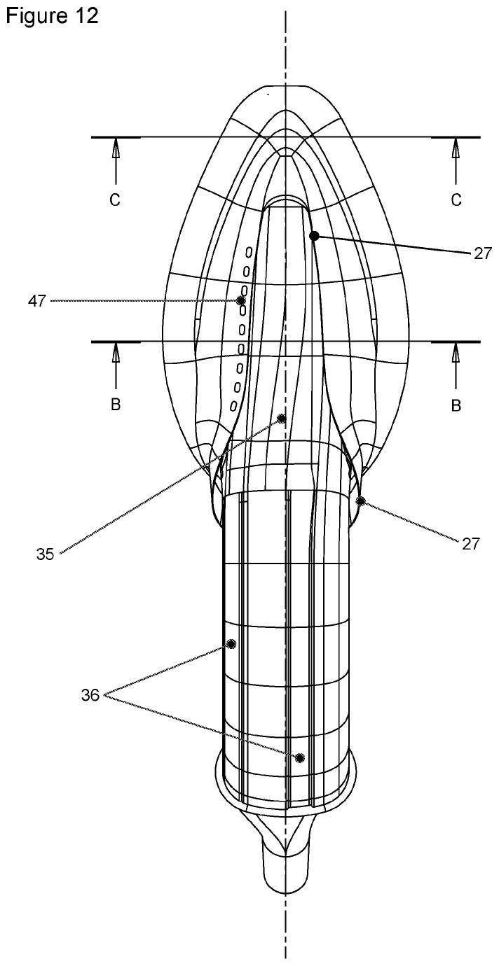

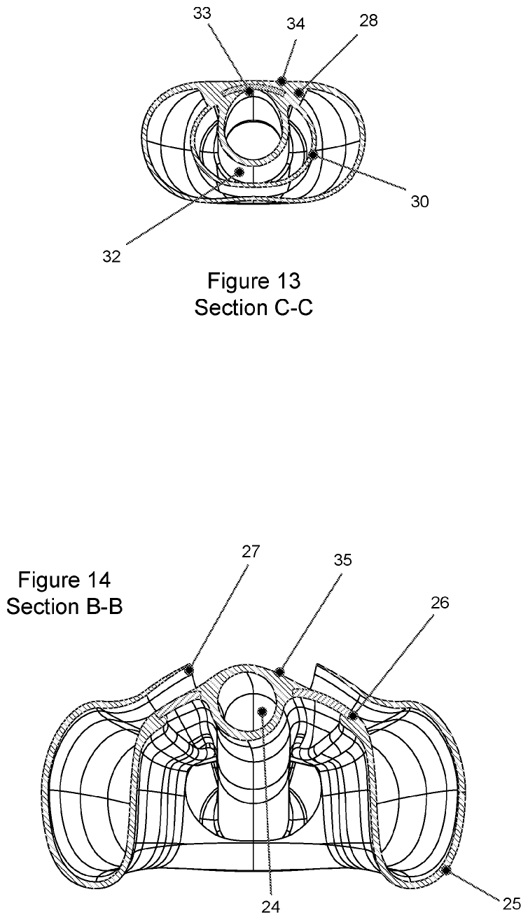

Horizontal cross sections B-B and C-C (FIGS. 13 and 14) illustrate the ventral or anterior opening 7 through which the primary and secondary passages exit. With respect to FIG. 14, the perimeter of the ventral opening is defined by a thin walled inflatable cuff membrane exhibiting an elliptical section 25. Adhered in the first instance to the perimeter of distal airway tube 26 and continuing tangentially from the immediate anterior of the airway tube toward its lateral extremity and normal to the edge of the airway tube. The method of manufacturing requires the cuff membrane to be open along the posterior opening 27 of the perimeter (FIGS. 12 and 14), except for a region surrounding the distal opening (FIG. 13) of the gastric drain tube that is moulded into a closed section 28 defining the configuration of the inflatable cuff surrounding the distal drain tube. To this end, as a result of the first injection moulding step, the inflatable portion or cuff is in the form of an open toroidal shape having the membrane open along a periphery of the toroid and adjacent to the periphery of the airway tube.

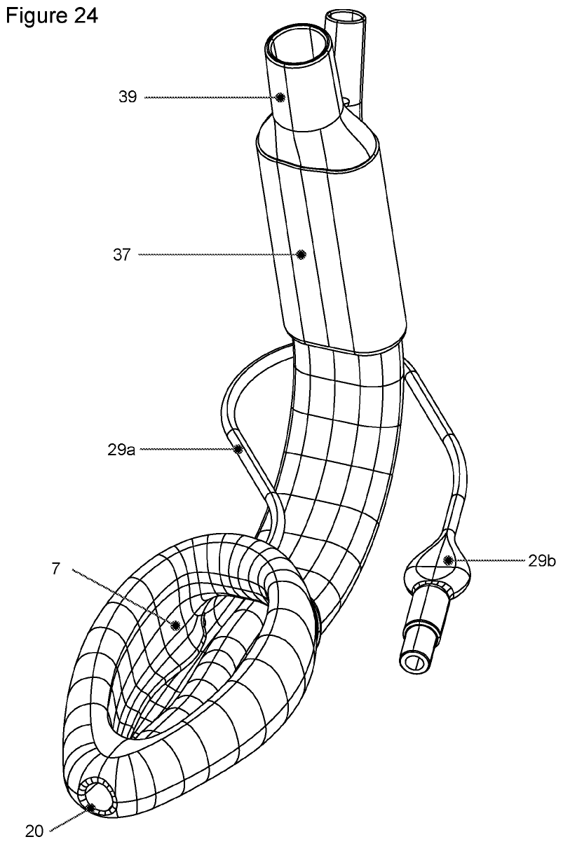

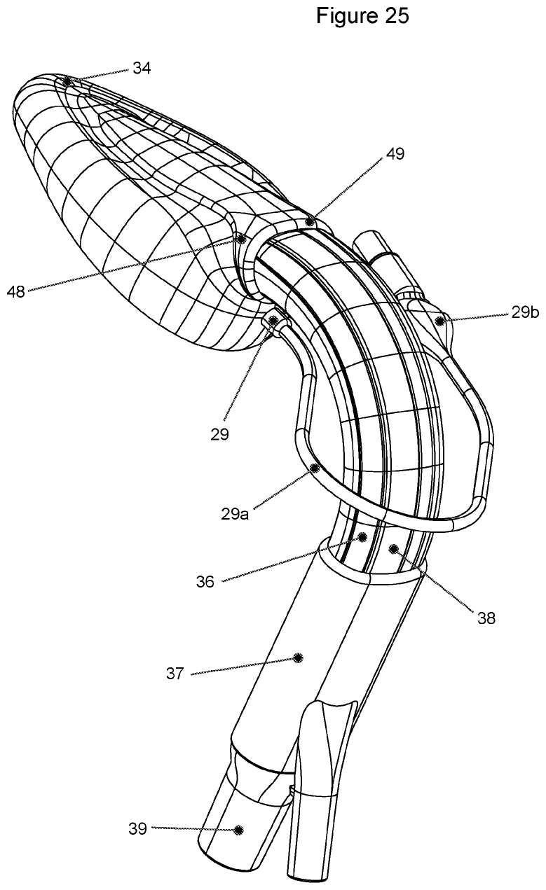

With reference to FIG. 8, the thickness of airway tube along the perimeter 26 varies from 1.00 at location 26a to 0.5 mm at location 26b combines with the compound curvature 33 at the distal airway tube to provide flexural articulation rather than flexure of the distal tip around a fixed horizontal axis. The thickness of the inflatable cuff membrane varies between 0.25 mm (leading edge of posterior opening 27) and 1.50 mm along the perimeter of the distal airway tube 26. All other cuff membrane wall thicknesses are optimised to provide for the ideal inflated shape and mechanical strength e.g. that portion of the inflatable cuff membrane (FIGS. 11, 20 and 21) surrounding a small tubular port 29 for attaching an inflation tube 29a extruded from thermoplastic elastomer (FIGS. 24 and 25) and equipped at its proximal end with an inflation balloon 29b and check valve allowing gaseous communication with the inflatable cuff.

In some embodiments, the distal portion of the gastric drainage tube may not intersect the inflated volume of the cuff (FIGS. 13 and 14). In this embodiment the outside diameter may not be directly exposed to the inflation pressure within the inflatable cuff; wall thickness of the gastric drainage requires no reinforcement structure to prevent occlusion; thereby avoiding a bulbous distal cuff configuration. Instead, and consistent with the closed section 28, the inflatable cuff membrane 25 is moulded into a closed tubular section 30 concentric with the third passage or gastric drain tube 24 creating a free space, or chamber, 32 adjacent to the tip of the device, and between the inflatable membrane and the distal third passage particularly about the aperture through which the gastric drainage tube projects. When in situ and inflated, the closed section of cuff membrane 30 will not expand to an extent that all free space or chamber 32 is eliminated and the gastric drain tube 24 compressed and occluded. The free space or chamber 32 therefore provides an expansion buffer, the size of which may be determined through design to accommodate sufficient inflation of the cuff. The cuff will therefore expand to within proximity, providing support to the third passage, or gastric drain tube 24, and the distal opening 20 against the upper oesophageal sphincter.

Furthermore, immediately superior to the distal opening, the anterior of distal airway tube compound curvature 33 defines the internal posterior surface of the third passage or gastric drain tube; the narrow width and curvature of the airway tube; the reducing thickness 26b and; the surrounding contour 34 of self-adhered TPE elastomer, minimise the deflated thickness of the distal tip. The elastic response of the polyolefin airway tube is manifest at the distal tip, now assisted by the softer TPE. This configuration keeps combined thickness of materials to a minimum, a characteristic evident when the cuff is deflated prior to deployment, negating the potentially bulbous nature of the distal cuff and gastric drainage supporting structure.

The contour of TPE adhering to the distal anterior airway tube (FIG. 13), having defined the closed section 28, progresses superiorly along the progressive curvature 31 of the medial slot 9, blending the resultant posterior contour 35 smoothly onto the intermediate strip, where it locates against the proximal end 8 of the medial slot. At this juncture the TPE diverts either side of the intermediate strip (FIGS. 7 and 12) filling the sagittal planar voids 36 defined by the intermediate strip locating against the posterior curvature of the airway tube 6. Proximally, at the juncture of the abruptly narrowing intermediate strip 19, the TPE 37 converges to surround the latches 18 and the intermediate strip in its entirety where it straddles the airway tube; the union of the intermediate strip and the airway tube is completed. Surrounded by TPE, the sealed union creates an enclosed third passage or gastric drain tube with proximal and distal opening.

The angle of the tubular feature relative to the adaptor (FIGS. 16 and 17) combined with the elastic nature of the TPE allows the user to; apply leverage to the tubular feature 12; in a direction 40 such that the angle of incidence through the retaining step 14 relative to the frontal plane (FIG. 9) is reduced and; remove the adaptor for insertion of an endotracheal tube or endoscope.

The adaptor can be returned to its original position by inserting the distal end into the proximal airway tube opening 42 and pushing it posteriorly. Once the notch 43 in the adaptor encounters the raised step 14 on the tubular feature 12; a moderate increase in pressure will enable the adaptor to snap back into the home position; the mating face 44 of the adaptor (FIG. 5) is pressed into and creates an airtight seal against the TPE 45 covering the proximal end 1 airway tube and intermediate strip and; a cylindrical cut-out 46 in the adaptor provides a minimal clearance against the tubular feature 12.

The subsequent injection moulding process provides a core and cavity that locates the leading edge of the open cuff membrane firmly against an airway portion such as the posterior distal airway tube perimeter 26. TPE interacts with the leading peripheral edges, entrapping them and blending with the already complete distal closed section 28 and conforming to the finished inflatable cuff contour defined by the injection mould core and cavity to close the toroidal cuff. A further embodiment (FIG. 12) of this interaction encourages the TPE to further entrap the leading edge via small cut-outs 47 and adhere it directly to the posterior of the distal airway tube.

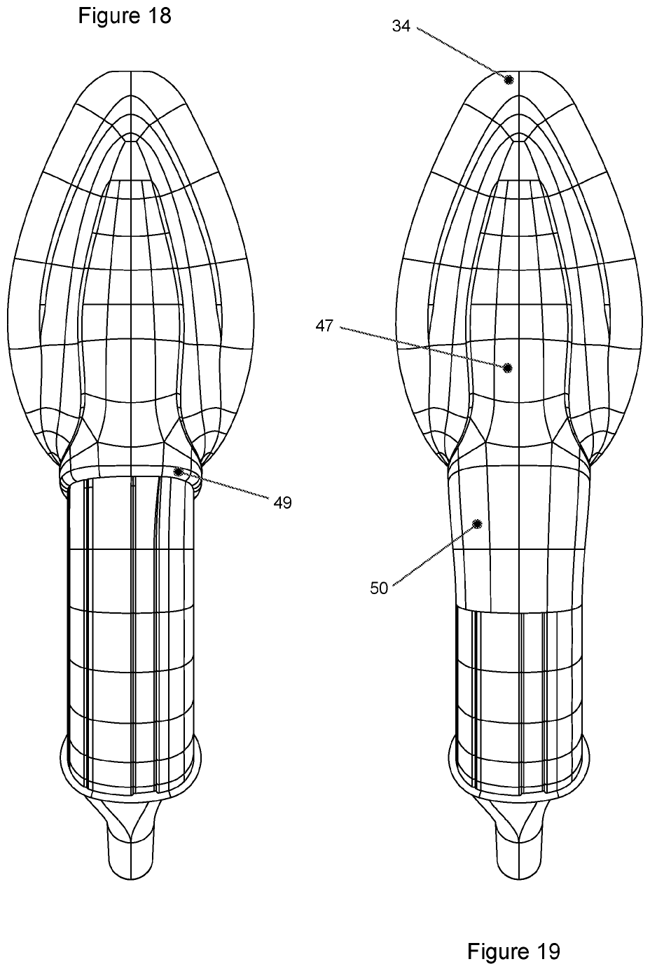

The finished contour of the distal portion (FIGS. 18, 20 and 22) adds additional TPE to the initial posterior contour 35 of the airway tube, wrapping around and completing a sealed circumference of the airway tube 48; creating an airtight inflatable cuff. A further embodiment of this circumferential blend (FIGS. 19, 21 and 23) shows the step 49 tapering away to a smooth blend 50 around the circumference of the airway tube.

The inflatable cuff membrane completes the manufacturing of the device described by this invention, without the need for adhesives or solvents. Using entirely polyolefin-based materials achieves a more ecological sustainable alternative to PVC and vinyl elastomers that may contain DEHP plasticisers or, LSR that cannot be recycled and similarly re-processed because it is a thermoset material whose cross-linking during moulding cannot be reversed.

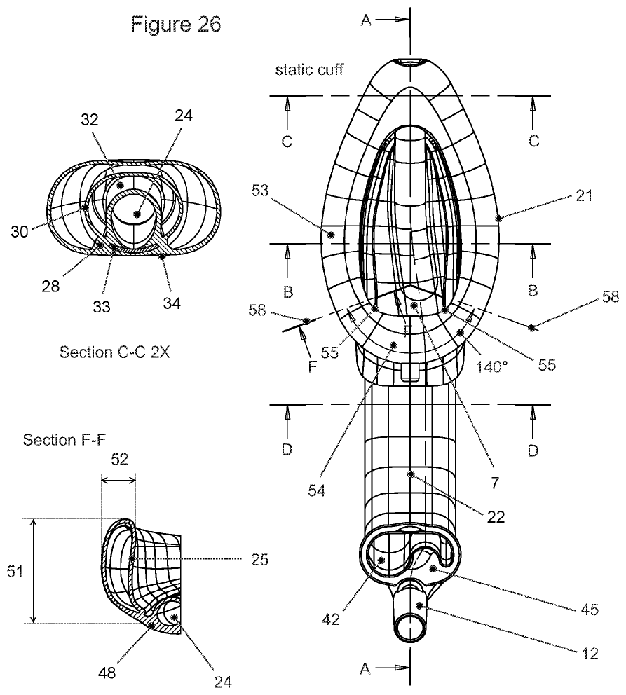

According to a further aspect of the disclosure, and with reference to FIGS. 26-34, in some embodiments, the body 6 may comprise a thin wall moulding of a majority of polypropylene random co-polymer, blended with a lesser amount of SEBS, the latter creating a dispersed elastomeric phase within the polypropylene (Abreu FOMS, Forte MMC, Liberman SA. SBS and SEBS block copolymers as impact modifiers for polypropylene compounds. Journal of Applied Polymer Science, Vol. 95,254-263 (2005)). This thin walled moulding is henceforth interchangeably referred to as an external shell when discussing the mechanics of the body as a lever, and an airway tube when referencing the function of the body 6 as a breathing conduit or passage.

Specifically, according to one embodiment, the initial posterior contour 35 may comprise SEBS over-moulded onto the external shell that flows into the cuff membrane 25 forming the inflatable cuff. Being mutually soluble, the SEBS diffuses into the polypropylene and likewise, the polypropylene diffusing into the SEBS creating an interphase of entangled polypropylene and SEBS along the perimeter of the distal airway tube 26 creating a first opposed edge. Simultaneously, a linear portion is over-moulded to the proximal end 37 such that the curved or initial posterior contour 35 and the linear portion 37 are joined as a single moulding by virtue of the sealing voids 36 to the left and right sides of the intermediate strip 38. Completing a first over-moulding without adhesive bonding or welding of discrete components, immediately followed by a second over-moulding of SEBS that seals the posterior open perimeter 27 or second opposed edge of the cuff membrane 25. The second over-mould covers the initial posterior contour 35 with additional SEBS closing and sealing the cuff membrane with the sealed circumference 48. The body 6, as described, has effectively been “assembled in the mould” using three separate injection moulding processes and is now complete with all required fluid communication passages and inflatable cuff.

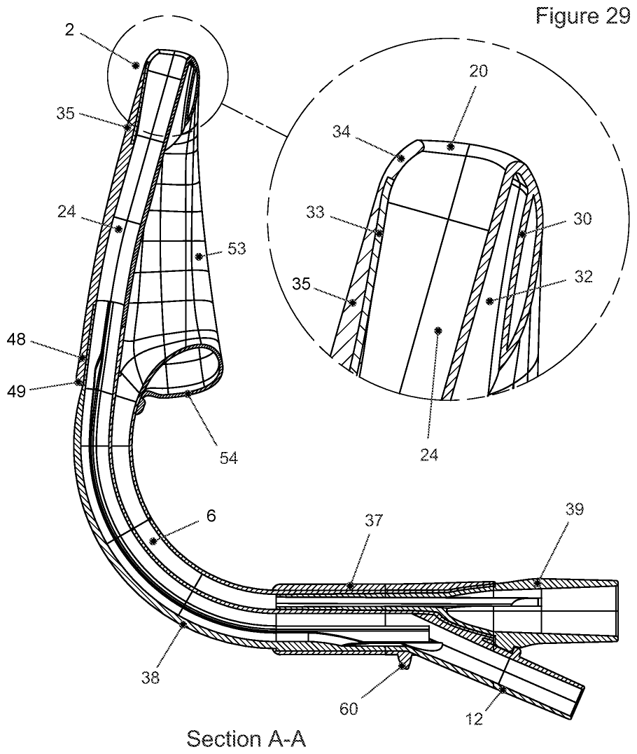

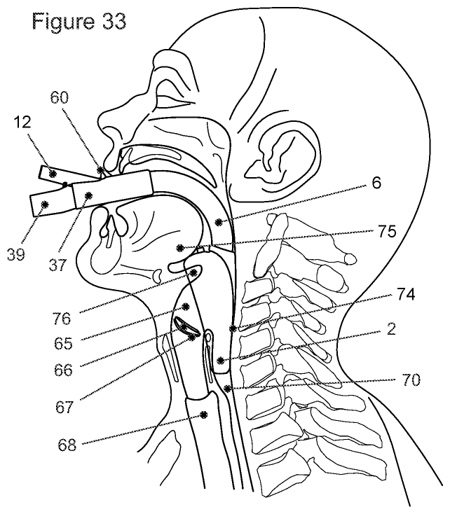

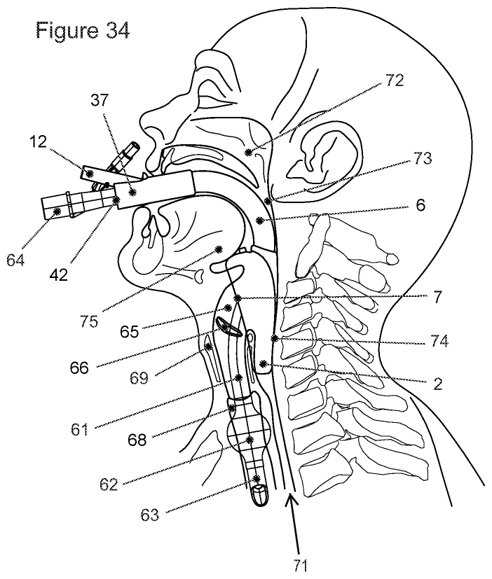

The inflatable cuff is used to seal the upper oesophageal sphincter 70, as shown by FIG. 33 but the distal portion of the inflatable cuff must include a mechanism that reduces compression of the glottic inlet along the anteroposterior axis when the distal end 2 of the body 6 is wedged into the upper oesophageal sphincter 70. A cross section through the distal end 2 is shown in FIG. 29. The distal posterior contour 34 of the distal end 2 over-moulds the compound curvature 33 of the external shell. The distal contour 34 of over-moulded TPE extends superiorly, blending into the initial posterior contour 35 and the distal perimeter of the external shell or first opposed edge 26. This extension or closed section 28 (FIG. 26 Section C-C) completes the over-moulded distal portion of the third passage or gastric drainage tube 24. The anterior of this compound curvature 33 defines the internal diameter and the route of the gastric drain tube 24; terminating at the distal opening 20. With the distal posterior contour 34 and closed section 28 located and pressed against the hypopharynx 74, displacement of this distal portion superiorly by increasing oesophageal pressure 71 (FIG. 34), is resisted by the distal to proximal full-length configuration and anatomical curvature of the body 6 within the pharynx. The softer distal posterior contour 34 and distal opening 20 wedged into the upper oesophageal sphincter, maintaining an effective oesophageal seal.

When the cuff is inflated, the gastric drain tube 24 and the drain tube distal opening 20 are not displaced anteriorly as they are integral with the distal posterior contour 34 pressed against the hypopharynx 74 i.e. the third passage or gastric drain tube 24 is not surrounded by an annular volume within the inflated cuff (FIG. 29) and in consideration thereof, does not require stiffening against occlusion by an expanding inflatable cuff. Only the distal portion of the cuff membrane moulded into a closed section 30 concentric to the gastric drain tube 24 is displaced anteriorly. The chamber 32 formed by the closed section 30 provides a space for said distal portion 30 to adapt to the anatomy with a posterior displacement when inflated, rather than pushing the glottis 66 anteriorly.

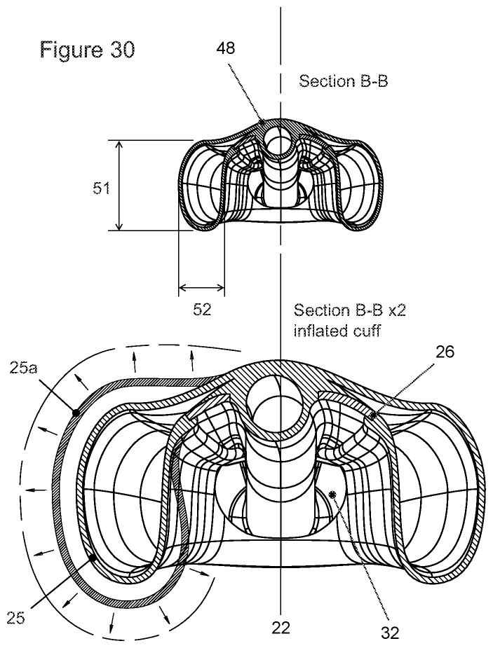

In this embodiment, the non-inflatable bulk of the body 6 within the pharynx is reduced because the required resilience and flexural response is provided by the thin walled external shell rather than an assembly of multiple components of differing hardness and thickness. The close proximity of (or gap between) all points describing the perimeter of the distal airway tube 26, normal to those describing the leading edge of the open cuff membrane 27, or first and second opposed edges respectively, is closed by the second over-mould creating the inflatable cuff. The cuff seals posteriorly against the hypopharynx 74 and anteriorly against the laryngeal inlet 65 by a single inflatable membrane 25 without adhesive joint (FIG. 30). Effectively maximising the surface area of inflatable cuff and minimising contact of non-inflatable surfaces (surfaces with no elastic recoil) to the anatomy and therefore, minimising direct compression of nervous structures by non-inflatable surfaces.

Neurological injury is multifactorial with the inflatable cuff being the significant contributing factor, either too rigid during insertion or direct compression of nervous structures whilst in situ. The cuff membrane 25 over-moulded and integral with the body 6 exhibits resilience and a measured elasticity during insertion. The cuff membrane can be deflated to present a flat wedge shape facilitating insertion between the teeth, past the tongue and through the palatoglossal arch. The ability of SEBS to stretch or elongate more than its original length for a given tensile force can be limited by the relative amounts of hard and soft domains within individual polymer strands; soft domains offering elasticity and conformance to the anatomy when inflated, the harder domains ensuring resilience and conformance to the as moulded shape. LSR or PVC elastomer, being single domain, tend to expand anterolateral when anteroposterior resistance is encountered, abandoning the as moulded shape and compressing nervous structures such as the lingual and hypoglossal nerve.

Cross section B-B (FIG. 30) through the lateral cuff reveals an under-square relationship of the height 51 to width 52, i.e., the height is always greater than the width. At low inflation pressure or when pressure inside and outside of the cuff membrane 25 is equal, this under-square relationship combined with the cuff membrane 25 maintaining the as moulded shape, creates an initial anatomical seal. When inflated 25a, the same under-square relationship is conserved ensuring reduced anterolateral expansion of the cuff.

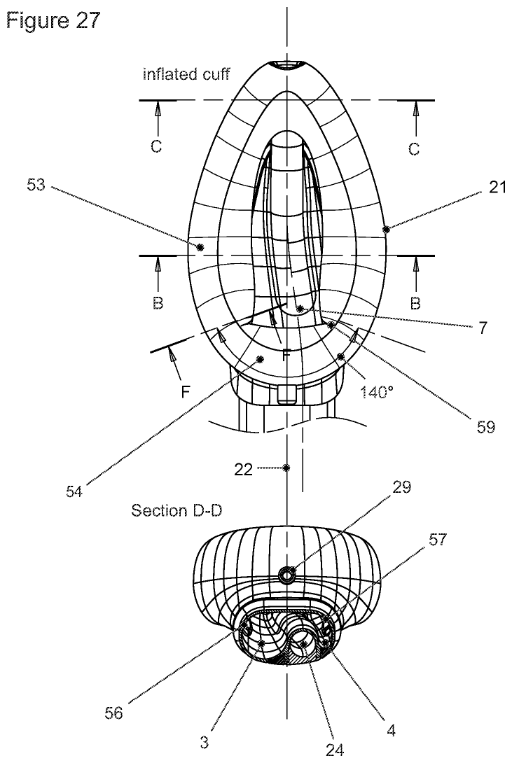

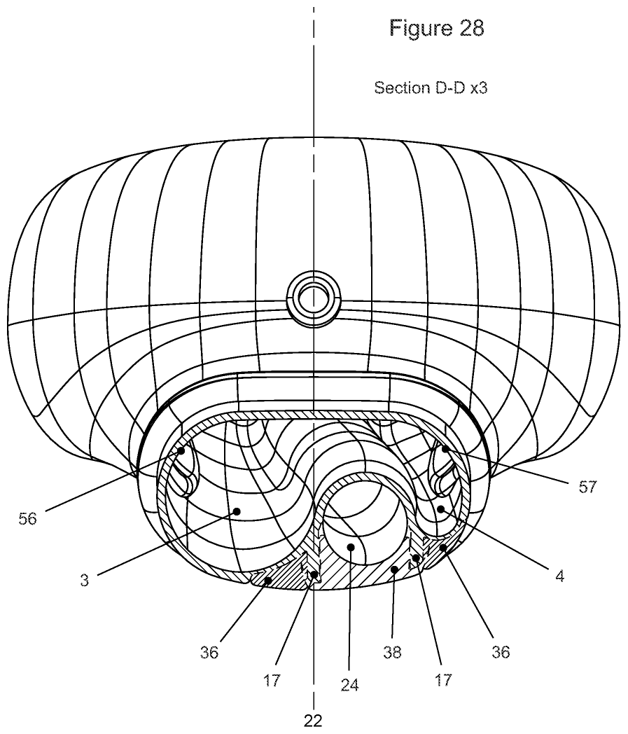

With reference to FIG. 26, curvature of each lateral extremity or curved perimeter 21 meets tangentially at their intersection with the median plane 22. Both lateral portions of the cuff 53 blend into the proximal portion 54, the ventral opening 7 adopting a rectangular configuration where each blend 55 is an arc whose radii are equal in magnitude; being a continuation of the internal radius of first passage 56 and the radius of second passage 57 respectively illustrated by FIG. 28. A line 58 passing through the centre of each radius blend 55 at 70 degrees either side of Section A-A creates a vertical angle of 140 degrees. Section F-F of FIGS. 26 and 27 is a planar section through one of the lines 58. The enclosed 2D sectional area of the cuff membrane 25 is the least of all other under-square sections through the cuff membrane. When inflated (FIG. 27), both lateral portions 53 and the proximal portion 54 expand, compressing against each other at this Section F-F such that each blend 55 becomes a fold 59 and maintaining the ventral opening 7 into the first bore and in so doing, limiting over-inflation of the proximal portion of the inflatable cuff 25 against the base of the tongue 75; the proximal portion of the inflatable cuff 25 maintaining alignment with and pressing against the tip of the epiglottis 76 (FIGS. 33 and 34).

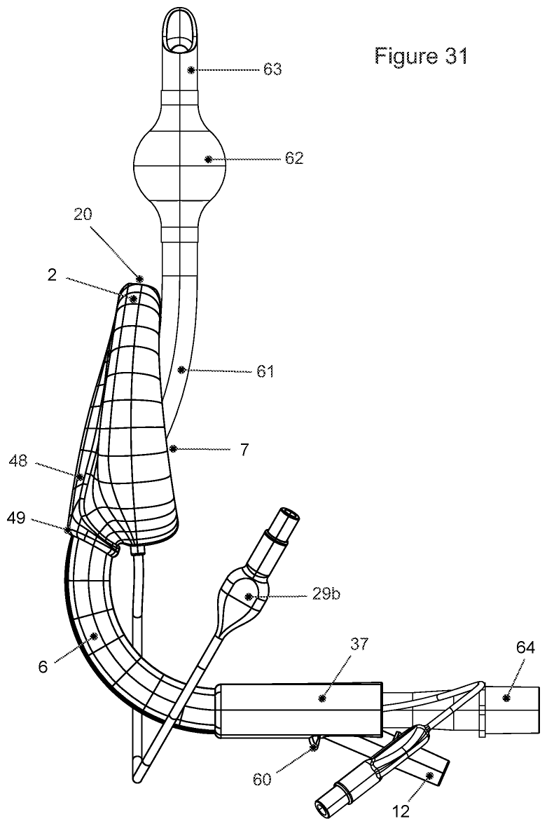

With the patient in the supine position (FIG. 34), intubation with an endotracheal tube (ETT) 61 requires the distal end 62 to exit the ventral opening 7 through the laryngeal inlet 65 and aligned with the glottis 66. As the ETT 61 continues inferiorly through the glottis, the distal end 62 of the ETT 61 must be correctly aligned with the trachea 68 (FIGS. 33 and 34). Overall, the length of the airway tube from the proximal opening 42 to ventral opening 7 should be kept to a minimum so that the distal end 62 of the ETT can be positioned sufficiently inferior to the glottis 66. To facilitate this minimum requirement, pressure is applied superiorly to the tubular port 12 to release the raised step 14 that retains the connector 39 (FIG. 31-33).

The most effective path or conduit for intubation and gastric drainage occupy the same anatomical space intermediate to the ventral opening 7 and the proximal opening for the combined primary and secondary passage 42. Their relative relationship contributes to the overall bulk of the device. With limited space available in the oropharynx 73 and to avoid pressure neuropraxia from the body 6, an anatomically approximating curvature is used with precedence given to the first or primary passage 3 occupying the space closest to the median plane 22 of the body 6, albeit slightly offset from said median plane (FIG. 28).

The third passage for gastric drainage is symmetrically opposite and parallel to the first passage 3, with the planar voids 36 on either side of the intermediate strip 38 defining this offset. Rather than just occupying interior space of the airway tube, the third passage for gastric drainage is a structural component, i.e., it contributes to the overall flexural strength of the external shell of the body 6, allowing the wall thickness of the primary passage 3 to be minimised in favour of maximum interior space for intubation. SEBS filling the planar voids 36 creates an interphase of entangled polypropylene and SEBS along the entire length of the anatomical curvature described by the supporting ribs 17 and intermediate strip 38. During insertion, flexion and extension applied to the proximal end of the body 6 are dissipated as shear, absorbed by the aforementioned interphase bonded to the intermediate strip 38 of the body 6.

The combined width of the body 6 may be symmetrical about the median plane 22. A nominal (e.g. 1.00 mm) wall thickness of the external shell reduces overall bulk and maximises the inside diameter for the first passage 3 allowing for an adult ETT 61 of a typical 8.5 mm inside diameter for a size 4 device. The removable connector/adaptor 39 reduces the length from the proximal opening 42 of the body 6 through to the trachea 68, providing additional depth of insertion of the ETT 61.

If an ETT 61 comprising a semi-rigid curved PVC tube is inserted into the proximal opening of the body 42 with the curvature of the tube orientated as if using a laryngoscope (ETT curvature follows the anatomical curvature) then the exit of the ETT distal tip 63 as it enters the laryngeal inlet 65 will be directed toward the thyroid cartilage 69 rather than the glottis 66. However, the exit trajectory of the ETT distal tip 63 from the first passage 3 through the ventral opening 7 and into the laryngeal inlet 65 can be optimised by lifting the proximal opening 42 of the body 6 anteriorly by gripping the receiving tube 12. The distal tip 61 of the ETT 62 can exit the ventral opening 7 and enter the laryngeal inlet 65 with closer alignment to the trachea 68 as shown in FIG. 34. As ETT's are predominantly manufactured from PVC and PVC being characterised as a viscous polymer, the force of bending the ETT shaft 61 through the primary or first passage 3 encourages a curvature whose equivalent radius is smaller than the pre-set curvature of the ETT shaft 61. The energy required to bend the ETT shaft 61 is dissipated into and through the length of the shaft. When the ETT distal tip 63 exits the ventral opening 7, the protruding distal tip 63 including the balloon 62, tries to return to its original curvature. However, recovery is not immediate.

This lost energy or delayed recovery can be advantageous toward alignment of the distal tip 63 with the glottis 66. As the ETT distal tip 63 enters further into the laryngeal opening, the lost energy is recovered allowing the shaft of the ETT 61 to partially straighten. During recovery, the body 6 can be raised anteriorly by gripping the receiving tube 12 to align the ETT distal tip 63 to the glottis 66. Thereafter, the ETT 61 is further inserted; the distal tip 63 passing through the vocal cords 67 and into the trachea 68. The connector/adaptor 64 is then removed from the tubular shaft of the ETT 61. The inflatable cuff 25 is deflated and the body 6 is removed, leaving the ETT in situ. Thereafter, the ETT connector/adaptor 64 is returned to its previous position. The viscous nature of the ETT tube body will allow a progressive and atraumatic recovery of curvature.

According to a further aspect of the disclosure, a method of manufacturing is also disclosed. As background, a conventional injection mould comprises a core that generally defines the concave or inside of the moulded component and a cavity that defines the convex or outside of the moulded component. Molten polymer is injected into the mould via a single screw/plunger mechanism. Allowed to cool and solidify, the polymer shrinks onto the core from whence it is removed. A second screw mechanism can be added so that polymer of two different colours or two different polymers can be injected into the mould, in most instances sequentially.

The requisite mould characterised by the complexity of moulding the initial component with the first polymer; then rotating the core by some inclusive mechanism to over-mould with the second polymer. Typically, the two cores are identical, but the corresponding cavities are different; the first cavity describes the substrate or base component geometry, the second cavity the final over-mould. Referred to as multiple component moulding, components separate to the process can also be introduced and over-moulded thus broadening the definition to in mould assembly. Technically, the scope of application using this method is limited to relatively small prismatic components and sub-assemblies because the application is confined within the physical constraint of a single injection moulding machine. Such complexity of moulds interacting in sequence can be described as a rigid body system, each mould requiring kinematic constraint i.e. the interaction between core and cavity for each mould set being a prismatic pair with a single degree of freedom (mould open and close) and the interaction between subsequent processes within the moulding machine frame being revolute pairs (core rotates to the next cavity when the mould opens). Both constraints characterised by a single degree of freedom.

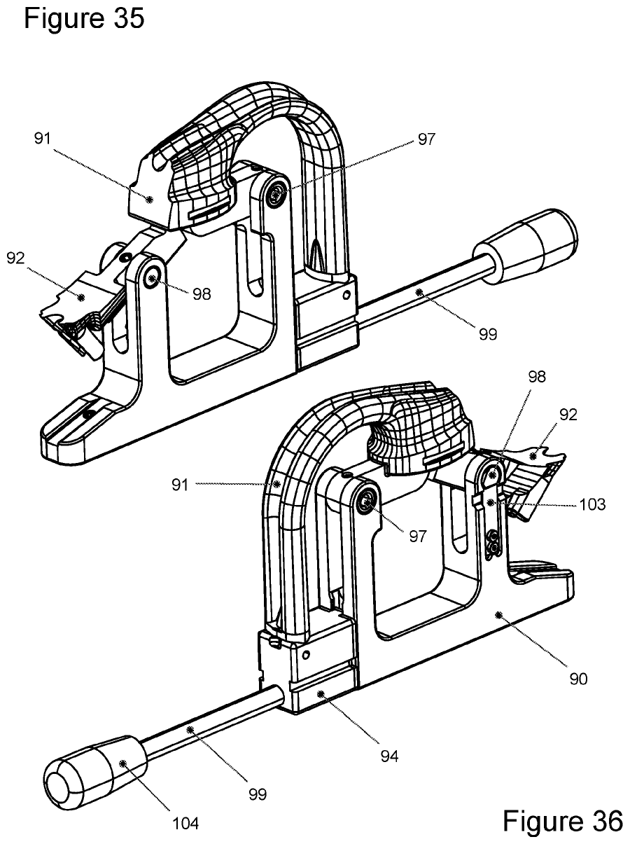

From the aforementioned embodiment, the synthesis of design features that reduce the characteristic bulk of the distal tip, reduce compression of the glottic inlet along the anteroposterior axis, reduce the risk of neurological injury and define the under square relationship of the inflatable cuff membrane 25, are realised by moulding cores, each core a rigid body and collectively a rigid body system. With reference to FIGS. 35 to 45, the fixture frame 90 is a constrained rigid body. Each mould core is joined, aligned or linked to the fixture frame 90 via a kinematic pair, or mechanism. Each kinematic pair refers to the kinematic constraints between each pair of rigid bodies that limits the motion of one rigid body with respect to the other. Constraints are planar or spatial degrees of freedom (DOF) i.e. linear or rotational displacement. An unrestrained rigid body has linear displacement in 3 axes and rotational displacement about each of these axes; a total of 6 DOF. The collective magnitudes of displacement for the kinematic pairs being sufficient to enable the completed body 6 to be removed from the rigid body system, henceforth referred to as the system.

The body 6 is not conceived of simple solid primitives displaying symmetry; the freeform geometric complexity of the passages or fluid paths within the body 6 requires moulding cores capable of linear and rotational displacement and combinations thereof, varying from one to six DOF. It is this complexity that sets it apart from multiple component moulding or in mould assembly. Rather than in mould assembly using a single injection moulding machine, body 6 is manufactured by transferring the system through a sequence of injection moulding machines. In this embodiment, three injection moulding machines (processes) are required. The external shell is the initial base or substrate component (first injection moulding process), the initial posterior contour 35, sagittal planar voids 36 together with the intermediate strip 38, and the linear portion 37 are the first over-mould (second injection moulding process). Subsequently, the initial posterior contour 35 and the planar voids 36 become the substrate and the sealed circumference 48 the second over-mould (third injection moulding process).

In mould assembly restricts the degrees of freedom related to mould configuration and therefore complexity of the moulded article, whilst creating an injection moulding process of significant complexity. To realise the design synthesis as described, the fixture frame 90 must be removable, transferable between injection moulding machines and in and of itself facilitate accurate interlock within each injection moulding process. Sequentially, within the space between moulding machines, additional components can be introduced, moulding cores added, removed or displaced by linear or rotational displacement or combinations thereof without the kinematic constraints inherent within the physical dimensions of a single moulding machine. The fixture frame 90 serves a datum reference for all processes until the completed device is removed from the fixture frame 90 using a demoulding mechanism. Essentially, the body 6 is assembled via the transfer of the system between injection moulding and non-injection moulding processes, each step of the assembly being an injection moulding process or assembly in the mould(s). The non-injection moulding processes allowing manipulation of the system mechanism and the introduction of external components using greater DOF than that possible by in mould assembly.

Beginning with FIGS. 35 and 36, a first rotating core 91 is a revolute pair constrained by a first pivot shaft 97. The revolute pair obeying a single DOF that permits the first rotating core 91 to rotate about an axis defined by the first pivot shaft 97. Similarly, a second rotating core 92 is a revolute pair constrained by a second pivot shaft 98. Proximal core 94 is a prismatic pair allowing linear displacement in one direction, and a single DOF is provided via slide mechanism shared with the fixture frame 90.

At the commencement of the manufacturing process and as illustrated by FIGS. 35 and 36, the rigid body system is placed into the first injection mould to mould the external shell. At this juncture, the rigid body system is in static equilibrium. The first rotating core 91 is constrained by the pivot shaft 97 and its rotation delimited by proximal core 94 and the second rotating core 92, whose position is fixed by a retainer, which may comprise an angular facet on the pivot shaft 98 bearing against the flat spring 103. When manually lifted by the handle 104, the mass of the system is supported by the proximal core 94 which bears against the first rotating core 91, which in turn bears against the second rotating core 92, fixed by the angular facet on a second pivot shaft 98 bearing against the flat spring 103.

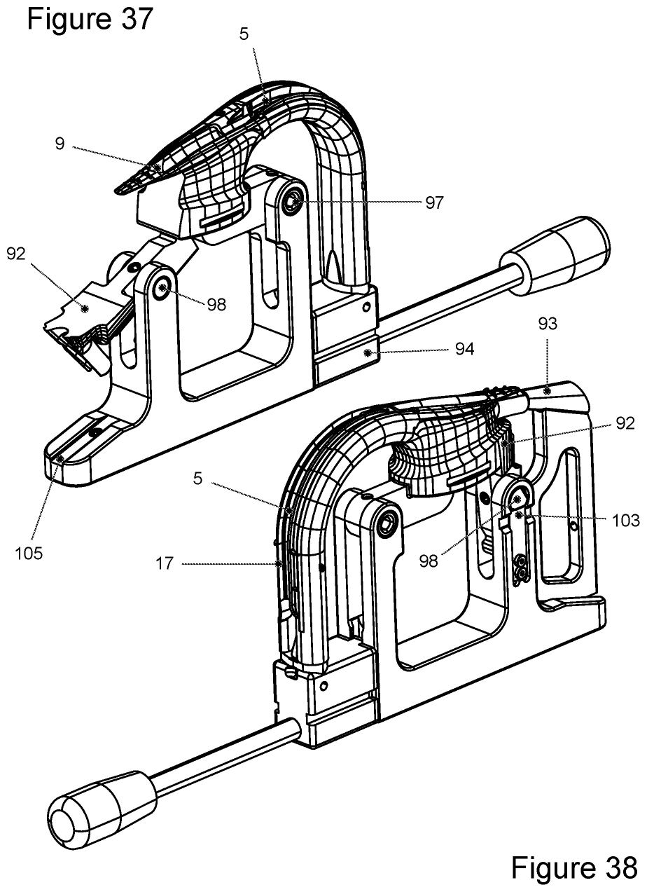

FIG. 37 shows the external shell moulded onto the first rotating core 91 and proximal core 94. After removal of the system from the first mould and prior to the placement in the second injection mould (second injection moulding process is the first over-mould), the second rotating core 92 is rotated to the position illustrated by FIG. 38 by releasing the flat spring 103. A distal core pin 93 is also introduced as an unrestrained rigid body in relation the fixture frame 90. This distal core pin 93 fits into an inclined channel 105 in the fixture frame 90 and locks into position. Note the supporting ribs 17 that will locate the intermediate strip 38.

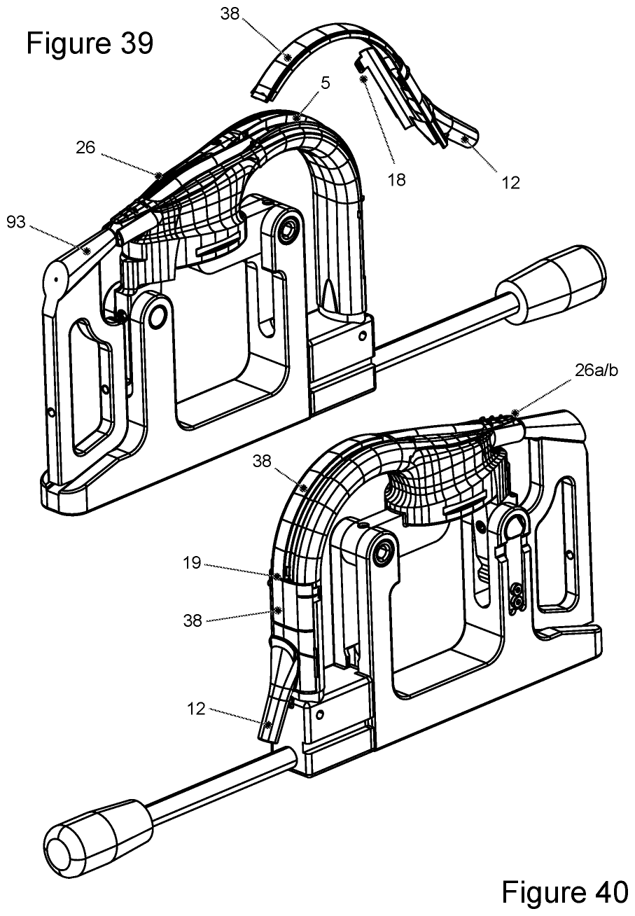

FIG. 39 illustrates the intermediate strip 38 orientated for attachment to the external shell. Latches 18 help to secure the intermediate strip 38 to the external shell. As the effective path for intubation and gastric drainage occupy the same space, precedence has been given to intubation and the partial third channel 5 (the intermediate portion of the third passage or gastric drainage tube 24) has been offset through the intermediate portion of the body. At the proximal and distal extremities of the third passage, the tubular port 12 and the distal tip 26a/b of the external shell are realigned with the central axis. This realignment and the angular configuration of the tubular port 12 relative to the partial posterior channel 5 prevent the use of a moulding core as there is no means of removing it. Hence, the unique feature of the intermediate strip 38 is that it is over-moulded without any means of resisting deformation from the heat and pressure of injection moulding other than the supporting ribs 17.

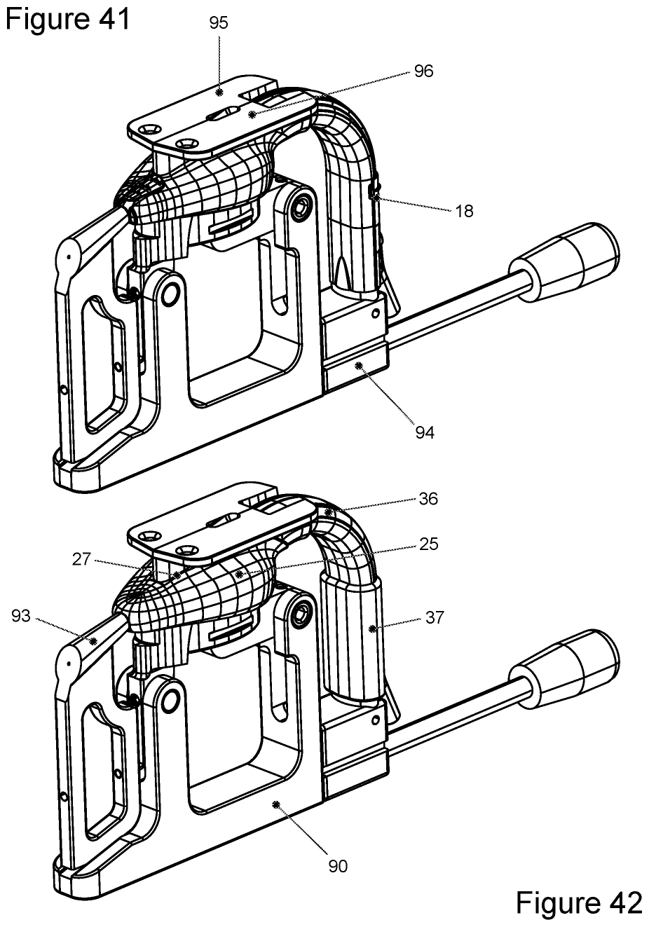

The system is placed into a second injection mould for the first over-mould. Unrestrained rigid bodies, such as a first removable core 95 and a second removable core 96, are also placed into the second injection mould. Closure of the mould locates the removable cores 95 and 96 in close proximity to the first rotating core 91 and second rotating core 92 as illustrated by FIG. 41. Subsequent over-moulding of the external shell becomes the body 6 complete with intermediate strip 38, the inflatable cuff membrane 25, the planar voids 36 and the linear portion 37 is shown by FIG. 42. Note the posterior opening 27 of cuff membrane 25. The system is then removed from the second injection mould.

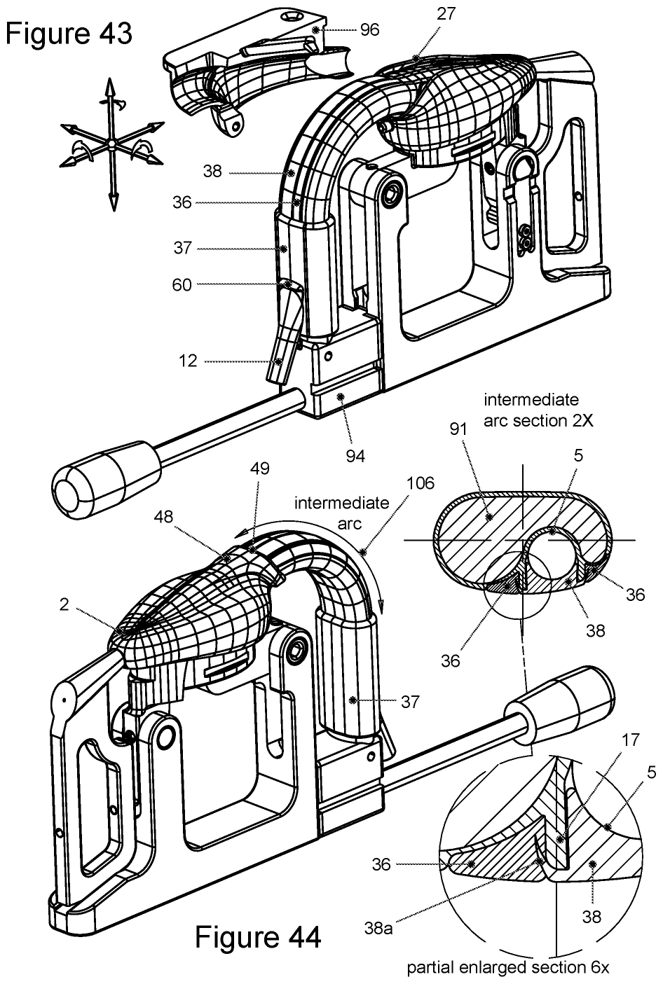

First removable core 95 and second removable core 96, when over-moulded with a material such as SEBS (forming the cuff membrane 25), are initially constrained by this membrane. In relation to the fixture frame 90, these removable cores 95 and 96 are unrestricted rigid bodies that are removed through the open cuff membrane by an external mechanism able to exploit the six degrees of freedom shown in FIG. 43.

Thereafter, the system is transferred to a third injection mould where the open cuff membrane 25 is closed up against the initial posterior contour 35 and subsequently over-moulded to create the sealed circumference 48 and 49 shown in FIG. 44. Additional embodiments of this sealed circumference are illustrated by FIGS. 18 to 22. This sealed circumference is the second over-mould. Future embodiments are not limited by the number of additional over-moulding processes because assembly in the mould is assembly via a sequence of moulding processes in separate injection moulding machines rather than in mould assembly, which implies numerous moulds within single injection moulding machine. The system is transferable availing itself to secondary processes without kinematic constraint.

As described above, the unique feature of the intermediate strip 38 is that it is over-moulded without any means of resisting deformation from the heat and pressure of injection moulding. To elucidate, the intermediate arc 106 shown in FIG. 44 represents the intermediate arc section of the body 6. The intermediate arc section 2X is common for any plane perpendicular to points along the intermediate arc and the centre point of the equivalent radius of the intermediate arc 106. The inside concave curvature or diameter of the intermediate strip 38 is identical to the cross-sectional diameter of the partial posterior channel 5 in the body 6; each intermediate arc section through the intermediate strip 38 being representative of a simply supported beam. During over-moulding, the sagittal planar voids 36 are filled with a material such as SEBS. The outer edges of the intermediate strip 38 being parallel to the sagittal planar voids 36 taper to a fine edge 38a. As illustrated by the partial enlarged section 6X of FIG. 44, these fine edges 38a act as a shield to protect the support ribs 17. Being sacrificial, the heat from first over-moulding process will melt the fine edges 38a of the polypropylene intermediate strip 38 diffusing it into the SEBS filling the sagittal planar voids 36. In addition, the pressure against the fine edge 38a presses it against the support rib 17, further sealing the third passage. The fine edges 38a also increase the over-mould surface area to better secure the intermediate strip 38.

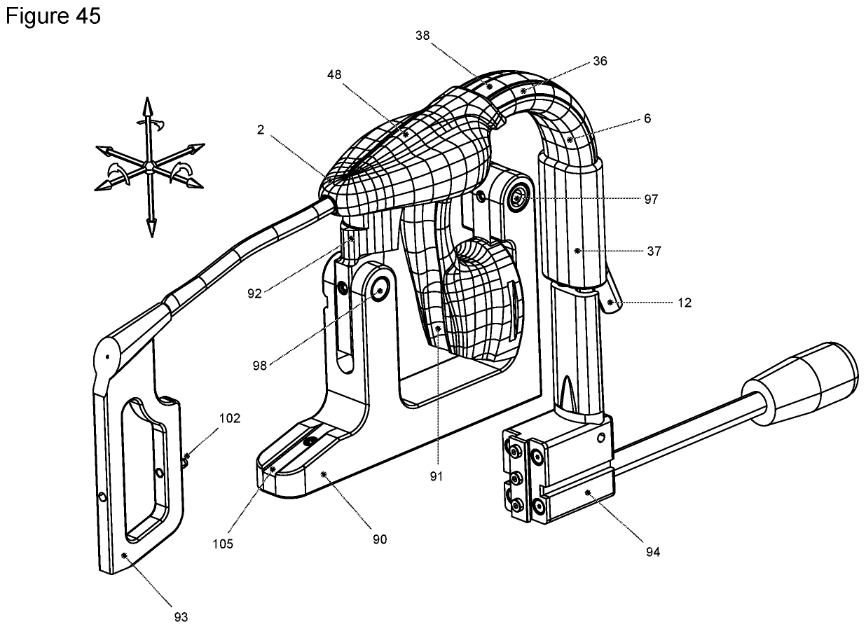

Finally, to remove the completed body 6 from the fixture frame 90, the individual cores defining each passage or fluid path, are functionally displaced following a finite path with respect to the fixture frame 90 which remains stationary. In FIG. 45, the first rotating core 91 rotates 90 degrees and proximal core 94 is displaced linearly to release the proximal end of the body 6. Distal core pin 93 is initially constrained by the over-moulded material exhibiting a planar pair. Surface treatment of the distal core pin 93 to reduce friction, enables transition to an unrestrained rigid body as it is removed from the body 6. Lastly, the body 6 remains attached to the second rotating core 92, the kinetic constraint being a cylindrical pair. Rotation around that portion of the second rotating core 92 that defines the closed tubular section 30 and a linear displacement along the axis of this tubular section 30 allows removal of the completed body. The constrained rigid body is returned to the configuration described by FIG. 35 and the process repeats.

In this embodiment, assembly in the mould is a kinematic synthesis of passages or fluid paths where passages are by design, functionally independent of each other and in combination, structurally dependent as one body. This synthesis is combination of material compatibility, design features and the unique manufacturing method as described.

Numbered Feature Index

| | |
|---|---|
| 1 | Proximal end of Body/ External Shell |
| 2 | Distal end of Body/ External Shell |
| 3 | Primary Passage |
| 4 | Secondary Passage |
| 5 | Partial Posterior Channel |
| 6 | Body exhibits an angle of curvature intermediate to proximal |
| | and distal ends |
| 7 | Ventral opening |
| 8 | Proximal end of Medial Slot |
| 9 | Medial Slot |
| 10 | Median Plane, Distal Tip |
| 11 | Cross Section through curvature intermediate to distal and |
| | proximal ends of Body |
| 11a | Ribs on Intermediate Strip that straddle the supporting ribs 17 |
| 12 | Tubular feature or port for gastric drain tube |
| 13 | Median axis of proximal Body |
| 13a | Median axis of Adaptor |
| 14 | Raised Step to retain Adaptor |
| 15 | Intermediate Strip transitions from tubular feature (port) |
| | elliptically straddling Body |
| 16 | Tubular contour opposite the elliptical transition 15 |
| 17 | Supporting Ribs that follow curvature of External Shell of the |
| | Body 6 locating Intermediate Strip 38 |
| 18 | Latches for attaching the Intermediate Strip |
| 19 | Immediate narrowing of the Intermediate Strip |
| 20 | Distal opening of the Body |
| 21 | Lateral extremities of curvature of Cuff/ Body |
| 22 | Median plane representing tangential closure of lateral |
| | curvatures 21 |
| 23 | Laterally opposed slots to capture stress concentration for |
| | flexion and extension |
| 24 | Third passage or Gastric Drain Tube enclosing the Medial Slot |
| | 9 |
| 25 | Cuff membrane |
| 26 | Perimeter of lateral portion of patient or distal end of External |
| | Shell |
| 26a, | Perimeter of distal portion of patient or distal end of External |
| 26b | Shell |
| 27 | Posterior opening of Cuff perimeter |
| 28 | Closed section of Cuff forming distal Gastric Drain Tube |
| 29 | Port for Inflation Tube |
| 29a | Inflation Tube |
| 29b | Inflation Balloon/ Check Valve Assembly |
| 30 | Closed tubular section concentric with third passage or |
| | Gastric Drain Tube 24 |
| 31 | Curvature defining the route of the Gastric Drain Tube 24 |
| 32 | Free space chamber between tubular section 30 and Drain |
| | Tube 24 |
| 33 | Compound curvature of distal airway tube |
| 33a | Anterior of distal airway tube compound curvature, parallel to |
| | the Medial Slot. |
| 33b | Anterior of distal airway tube compound curvature, |
| | perpendicular to the Medial Slot. |
| 34 | Distal posterior contour of TPE adjacent to the Distal Opening |
| | 20 |
| 35 | Initial posterior contour of TPE integral with Cuff membrane |
| | 25 |
| 36 | Sagittal planar voids joining over-moulding intermediate strip |
| | 38 to body 6. |
| 37 | Proximal end of Body, TPE covering Intermediate Strip |
| | straddling the External Shell |
| 38 | Intermediate Strip |
| 38a | Fine edge of Intermediate Strip |
| 39 | Connector/ Adaptor |
| 40 | Direction of leverage to release the Raised Step 14 from the |
| | Adaptor Notch 40 |
| 41 | Distal end of Adaptor mates with the proximal opening for the |
| | Adaptor in the Body |
| 42 | Proximal opening, the combined Primary and Secondary |
| | Passage |
| 43 | Notch in Adaptor to accept Raised Step 14 |
| 44 | Mating face of the Adaptor to the over-moulded proximal end |
| | of the Body 45 |
| 45 | TPE over-moulded proximal end of Body against which the |
| | Adaptor creates a seal |
| 46 | Cylindrical cut-out to clear Tubular Feature 12 |
| 47 | Additional embodiment for over-moulding Sealed |
| | Circumference 48 |
| 48 | Sealed circumference of TPE over-moulding the initial |
| | posterior contour 35 |
| 49 | Moulded edge defining sealed circumference around external |
| | shell |
| 50 | Additional embodiment, sealed circumference 48 blends |
| | smoothly into intermediate portion of body |
| | 6 without moulded edge 49 |
| 51 | Height of cuff through Cross Section B-B |
| 52 | Width of cuff through Cross Section B-B |
| 53 | Lateral portion of the cuff |
| 54 | Proximal portion of the cuff |
| 55 | Blend between lateral and proximal cuff creating an arc |
| 56 | Internal radius of first passage |
| 57 | Internal radius of second passage |
| 58 | Line either side of Section A-A creating a vertical pair. |
| 59 | Fold created by inflated lateral and proximal cuff compressing |
| | against each other |
| 60 | Finger stop |
| 61 | Endotracheal tube (ETT) |
| 62 | ETT balloon |
| 63 | ETT distal tip |
| 64 | ETT Connector/ Adaptor |
| 65 | Laryngeal Inlet |
| 66 | Glottis |
| 67 | Vocal Cords |
| 68 | Trachea |
| 69 | Thyroid Cartilage |
| 70 | Upper Oesophageal Sphincter |
| 71 | Increasing oesophageal pressure |
| 72 | Nasopharynx |
| 73 | Oropharynx |
| 74 | Hypopharynx |
| 75 | Tongue |
| 76 | Epiglottis |
| 90 | Fixture Frame |
| 91 | First Rotating Core |
| 92 | Second Rotating Core |
| 93 | Distal Core Pin |
| 94 | Proximal Core |
| 95 | First Removable Core |
| 96 | Second Removable Core |
| 97 | First Pivot Shaft |
| 98 | Second Pivot Shaft |
| 99 | Handle Shaft |
| 100 | Spring Pin |
| 101 | Ball Plunger |
| 102 | Dowel Pin |
| 103 | Flat Spring |
| 104 | Stationary Handle |
| 105 | Inclined channel |
| 106 | Intermediate arc |

This disclosure may be considered to be related to any or all of the foregoing items in any combination:

1. An airway management device, comprising:

a body (6) including an external shell moulded from a polypropylene copolymer (PP) blended with a thermoplastic elastomer (TPE) of styrene-ethylene/butylene-styrene (SEBS), the external shell extending from a proximal opening to a distal tip of the body (6), the external shell having a curved portion (35) and a linear portion (37).

2. The airway management device of item 1, further including an intermediate strip (38) moulded from a polypropylene copolymer (PP) attached to said external shell intermediate to the curved portion (35) and the linear portion (37).
3. The airway management device of item 1 or item 2, further including a first over-mould of SEBS comprising a posterior contour (35) and a distal contour (34) on the external shell.
4. The airway management device of any of items 1-3, wherein a or the first over-mould comprises a distal perimeter (26) defining a first opposed edge of an over-moulded cuff membrane (25) continuing tangentially from said perimeter (26) as a toroidal curve whose end point is in spaced relation and normal to the first opposed edge, the end points defining an open posterior (27) perimeter or second opposed edge and a linear portion (37) over-moulding a proximal end such that said curved portion (35) and the linear portion (37) are joined as a single moulding by planar sealing voids (36) to first and second sides of an intermediate strip (38).
5. The airway management device of any of items 1-4, further including a second over-mould of SEBS closing said open length of membrane (27), forming an inflatable cuff.
6. The airway management device according to any of items 1-5, wherein a or the posterior contour (35) of the body (6) is adapted to be located within a hypopharynx and a distal end (2) is adapted to be located within an upper oesophageal sphincter creating an oesophageal seal, wherein immediately superior to a distal opening (20), and the anterior compound curvature (33) of the external shell is an internal posterior surface of a passage or gastric drain tube (24) reducing the bulk of the distal tip (2).
7. The airway management device according to any of items 1-6, further including a surrounding contour (34) over-moulding said anterior compound curvature (33) of the external shell and which is adapted for locating and pressing against the hypopharynx, the distal to proximal full length configuration of the external shell providing resistance against displacement of the distal opening (20) superiorly from increasing oesophageal pressure (70).
8. The airway management device of any of items 1-7, wherein a or the drain tube (24) and a drain tube distal opening (20) are integral with a distal posterior contour (34) and where the said drain tube (24) is not surrounded by an annular volume of said inflatable cuff.
9. The airway management device of any of items 1-8, further including a closed tubular section (30) forming a chamber (32) providing a space for a distal portion of the inflatable cuff with a posterior displacement when inflated.
10. The airway management device of any of items 1-9, wherein through any horizontal cross section of said inflatable cuff, first and second edges (27), for at least the length of the distal portion of the gastric drain tube (31), are maintained parallel to a median plane (10) of the curved portion (35) such that the width between the first and second edges (27) after second over-moulding is equal to an outer diameter of the distal drain tube (31).
11. The airway management device of any of items 1-10, wherein a curvature of the inflatable cuff membrane (25) between said first (26) and second (27) opposed edges is a single contiguous curve of uniform durometer hardness, sealing posteriorly against a hypopharynx and anteriorly against a laryngeal inlet without adhesive joint.
12. A method of using the airway management device according to any of items 1-11, comprising:

providing a removable connector/adaptor (39) on the linear portion to reduce a length from a proximal opening (42) of the body (6) through to a trachea (68), thereby providing additional depth of insertion of a distal tip (63) of an endotracheal tube.

13. A method of using the airway management device according to any of items 1-11, comprising:

providing a finger stopper (60) creating a fixed position to rest a thumb during insertion, to grip a proximal end (37) when removing the device after intubation and to act as a depth indicator, with reference to teeth, when the device is in situ.

14. A method of forming an airway management device, comprising:

providing a body (6) comprising polypropylene copolymer (PP) and thermoplastic elastomer (TPE) of styrene-ethylene/butylene-styrene (SEBS), including an external shell moulded from a majority PP copolymer blended with SEBS extending from a proximal opening to a distal tip of the body (6).

15. The method of item 14, further including the step of:

attaching an intermediate strip (38) moulded from PP copolymer to said external shell intermediate to a curved portion (35) and a linear portion (37) of the body (6).

16. The method of item 14 or item 15, further including the step of:

providing a first over-mould of SEBS comprising an initial posterior contour (35) and distal contour (34) over-moulded onto the external shell, whose distal perimeter (26) defines a first opposed edge of an over-moulded cuff membrane (25) that continues tangentially from said distal perimeter (26) as a toroidal curve whose end point is in spaced relation and normal to the first opposed edge, the end points collectively defining an open posterior (27) perimeter or second opposed edge and a linear portion over-moulding the proximal end (37) such that said curved portion (35) and the linear portion (37) are joined as a single moulding by planar sealing voids (36) to lateral sides of said intermediate strip (38).

17. The method of item 16, further including the step of providing a second over-mould of SEBS closing said membrane (25), forming an inflatable cuff and completing the said body (6).
18. A method of forming an object, comprising:

injection moulding a first portion of the object over a first core associated with a fixture;

after the injection moulding of the first portion, moving a second core associated with the fixture to a deployed position; and

injection moulding a second portion of the object over the second core and the first portion.

19. The method of item 18, wherein the step of moving the second core associated with the fixture to the deployed position comprises rotating the second core relative to the fixture.
20. The method of item 18 or item 19, further including the steps of:

attaching a pre-formed part to the object;

placing one or more removable cores into the object; and

placing one or more removable cores inside the injection mould prior to moulding a second portion of the object; and

over-moulding a membrane as part of the second portion of the object.

21. The method of any of items 18-20, further including the step of injection moulding a third portion closing and sealing the membrane to form an inflatable portion of the object.
22. The method of any of items 18-21, further including the step of removing the removable cores from the membrane of the object prior to injection moulding the third portion.
23. The method of any of items 18-21, wherein:

the step of injection moulding the first portion is completed in a first mould including the fixture; and

the step of injection moulding the second portion is completed in a second mould including the fixture; and

the step of injection moulding the third portion to close and seal the membrane in a third mould including the fixture.

24. The method of any of items 18-23, further including the step of transferring the fixture from a first mould to a second mould between the steps of injection moulding the first portion and second portion of the object.
25. The method of any of items 18-24, wherein the step of injection moulding the first portion of the object over the first core associated with the fixture comprises forming the external shell of the object.
26. The method of any of items 18-25, further including the steps of:

moving the first core to release a proximal end of the object; and

removing the object from the second core of the fixture.

27. The method of any of items 18-26, further including the step of:

providing a body comprising polypropylene copolymer (PP) and thermoplastic elastomer (TPE) of styrene-ethylene/butylene-styrene (SEBS), and wherein the first portion comprises an external shell moulded during the first injection moulding step from a majority PP copolymer blended with SEBS extending from a proximal opening to a distal tip of the body.

28. The method of any of items 18-27, wherein the object comprises an airway management device.
29. An apparatus for forming an injection moulded object, comprising:

a reconfigurable fixture including a first movable core over which a first portion of the injection moulded object is formed and a second movable core over which a second portion of the injection moulded object is formed.

30. The apparatus of item 29, wherein the first movable core is adapted for rotating relative to the fixture.
31. The apparatus of item 29 or item 30, wherein the second movable core is adapted for rotating relative to the fixture.
32. The apparatus of any of items 29-31, further including a first removable core adapted for being removably attached to the fixture.
33. The apparatus of item 32, wherein the first removable core comprises a connector for connecting to the fixture.
34. The apparatus of item 32, wherein the first removable core comprises a handle.
35. The apparatus of any of items 29-34, wherein the fixture comprises a spring for maintaining the second movable core in a deployed position.
36. A method of manufacturing an airway management device, comprising:

providing a tubular body having a linear portion and a curved portion, the tubular body including a plurality of supports adjacent to a posterior channel;

providing an intermediate strip in engagement with the plurality of supports and overlying the posterior channel; and

over-moulding material onto the intermediate strip.

37. The method of item 36, further comprising the steps of:

injection moulding a first portion of the body of the airway management device over a first core associated with a fixture;

after the injection moulding of the first portion, moving a second core associated with the fixture to a deployed position; and

injection moulding a second portion of the body over the second core and the first portion of the body.

38. The method of item 37, wherein the step of moving the second core associated with the fixture to the deployed position comprises rotating the second core relative to the fixture.
39. The method of any of items 36-38, further including the steps of:

placing one or more removable cores in the tubular body; and

placing one or more removable cores inside the second injection mould; and

over-moulding the removable cores with a membrane as part of the second portion of the body.

40. The method of item 39, further including the steps of:

removing said removable cores from close proximity to the first core and second core leaving an open membrane; and

over-moulding the second portion with a third portion closing and sealing the membrane to form an inflatable cuff on the tubular body.

41. The method of claim 39 or item 40, further including the step of moving the first core and removing the removable cores to release the tubular body.
42. The method of any of items 36-41, further including the steps of:

placing a first material in one or more voids adjacent the intermediate strip with a first material; and

melting a portion of the intermediate strip comprising a second material so as to diffuse the first material into the second material.

43. A method of forming an object, comprising:

in a first injection mould, injection moulding a first portion of the object over a first core associated with a fixture;

placing the fixture in a second injection mould; and

injection moulding a second portion of the object.

44. The method of item 43, wherein the first core is movable relative to the fixture, and further including the step of moving the first core following the injection moulding of the first portion or the second portion of the object.
45. The method of item 43 or item 44, wherein the step of injection moulding the second portion of the object comprises injection moulding over a second core associated with the fixture.
46. The method of any of items 43-45, wherein the second core is movable relative to the fixture, and further including the step of moving the second core to a deployed position after the step of injection moulding the first portion of the object and prior to the injection moulding of the second portion of the object.
47. The method of any of items 43-46, further including the step of placing one or more removable cores inside the second injection mould prior to the step of injection moulding the second portion of the object.
48. The method of any of items 43-47, wherein the step of injection moulding the second portion of the object comprises over-moulding a membrane onto the one or more removable cores.
49. The method of any of items 43-48, wherein the one or more removable cores are removed from the second injection mould together with the fixture.
50. The method of any of items 43-49, wherein the one or more removable cores are removed from the membrane after injection moulding the second portion and prior to injection moulding the third portion of the object.
51. The method of item 50, further including the step of closing and sealing the membrane to form an inflatable portion of the object.
52. An airway management device formed by the method of any of items 36-51.

Each of the following terms written in singular grammatical form: “a”, “an”, and the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or the context clearly dictates otherwise. For example, the phrases: “a unit”, “a device”, “an assembly”, “a mechanism”, “a component, “an element”, and “a step or procedure”, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated components), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase “consisting essentially of.” Each of the phrases “consisting of” and “consists of”, as used herein, means “including and limited to”. The phrase “consisting essentially of” means that the stated entity or item (system, system unit, system sub-unit device, assembly, sub-assembly, mechanism, structure, component element or, peripheral equipment utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional feature or characteristic” being a system unit system sub-unit device, assembly, sub-assembly, mechanism, structure, component or element or, peripheral equipment utility, accessory, or material, step or procedure, sub-step or sub-procedure), but only if each such additional feature or characteristic” does not materially alter the basic novel and inventive characteristics or special technical features, of the claimed item.

The term “method”, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Terms of approximation, such as the terms about, substantially, approximately, generally, etc., as used herein, refer to ±10% of the stated numerical value or as close as possible to a stated condition.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

1. An airway management device, comprising:
a body (6) including an external shell moulded from a polypropylene copolymer (PP) blended with a thermoplastic elastomer (TPE) of styrene-ethylene/butylene-styrene (SEBS), the external shell extending from a proximal opening to a distal tip of the body (6), the external shell having a curved portion (35) and a linear portion (37).

2. The airway management device of claim 1, further including an intermediate strip (38) moulded from a polypropylene copolymer (PP) attached to said external shell intermediate to the curved portion (35) and the linear portion (37).

3. The airway management device of claim 1, further including a first over-mould of SEBS comprising a posterior contour (35) and a distal contour (34) on the external shell.

4. The airway management device of claim 3, wherein the first over-mould comprises a distal perimeter (26) defining a first opposed edge of an over-moulded cuff membrane (25) continuing tangentially from said perimeter (26) as a toroidal curve whose end point is in spaced relation and normal to the first opposed edge, the end points defining an open posterior (27) perimeter or second opposed edge and a linear portion (37) over-moulding a proximal end such that said curved portion (35) and the linear portion (37) are joined as a single moulding by planar sealing voids (36) to first and second sides of an intermediate strip (38).

5. The airway management device of claim 4, further including a second over-mould of SEBS closing said open length of membrane (27), forming an inflatable cuff.

6. The airway management device of claim 5, wherein the posterior contour (35) of the body (6) is adapted to be located within a hypopharynx and a distal end (2) is adapted to be located within an upper oesophageal sphincter creating an oesophageal seal, wherein immediately superior to a distal opening (20), and the anterior compound curvature (33) of the external shell is an internal posterior surface of a passage or gastric drain tube (24) reducing the bulk of the distal tip (2).

7. The airway management device of claim 6, further including a surrounding contour (34) over-moulding said anterior compound curvature (33) of the external shell and which is adapted for locating and pressing against the hypopharynx, the distal to proximal full length configuration of the external shell providing resistance against displacement of the distal opening (20) superiorly from increasing oesophageal pressure (70).

8. The airway management device of claim 6, wherein the drain tube (24) and a drain tube distal opening (20) are integral with a distal posterior contour (34) and where the said drain tube (24) is not surrounded by an annular volume of said inflatable cuff.

9. The airway management device of claim 6, further including a closed tubular section (30) forming a chamber (32) providing a space for a distal portion of the inflatable cuff with a posterior displacement when inflated.

10. The airway management device of claim 6, wherein through any horizontal cross section of said inflatable cuff, first and second edges (27), for at least the length of the distal portion of the gastric drain tube (31), are maintained parallel to a median plane (10) of the curved portion (35) such that the width between the first and second edges (27) after second over-moulding is equal to an outer diameter of the distal drain tube (31).

11. The airway management device of claim 10, wherein a curvature of the inflatable cuff membrane (25) between said first (26) and second (27) opposed edges is a single contiguous curve of uniform durometer hardness, sealing posteriorly against a hypopharynx and anteriorly against a laryngeal inlet without adhesive joint.

12. A method of forming the airway management device according to claim 1, comprising:
providing a removable connector/adaptor (39) on the linear portion to reduce a length from a proximal opening (42) of the body (6) through to a trachea (68), thereby providing additional depth of insertion of a distal tip (63) of an endotracheal tube.

13. A method of forming the airway management device according to claim 1, comprising:
providing a finger stopper (60) creating a fixed position to rest a thumb during insertion, to grip a proximal end (37) when removing the device after intubation and to act as a depth indicator, with reference to teeth, when the device is in situ.

14. A method of forming an airway management device, comprising:
providing a body (6) comprising polypropylene copolymer (PP) and thermoplastic elastomer (TPE) of styrene-ethylene/butylene-styrene (SEBS), including an external shell moulded from a majority PP copolymer blended with SEBS extending from a proximal opening to a distal tip of the body (6).

15. The method of claim 14, further including the step of:
attaching an intermediate strip (38) moulded from polypropylene copolymer to said external shell intermediate to a curved portion (35) and a linear portion (37) of the body (6).

16. The method of claim 15, further including the step of:
providing a first over-mould of SEBS comprising an initial posterior contour (35) and distal contour (34) over-moulded onto the external shell, whose distal perimeter (26) defines a first opposed edge of an over-moulded cuff membrane (25) that continues tangentially from said distal perimeter (26) as a toroidal curve whose end point is in spaced relation and normal to the first opposed edge, the end points collectively defining an open posterior (27) perimeter or second opposed edge and a linear portion over-moulding the proximal end (37) such that said curved portion (35) and the linear portion (37) are joined as a single moulding by planar sealing voids (36) to lateral sides of said intermediate strip (38).

17. The method of claim 16, further including the step of:
providing a second over-mould of SEBS closing said membrane (25), forming an inflatable cuff and completing the said body (6).

18. A method of forming an object, comprising:
injection moulding a first portion of the object over a first core associated with a fixture;
after the injection moulding of the first portion, moving a second core associated with the fixture to a deployed position; and
injection moulding a second portion of the object over the second core and the first portion.

19. The method of claim 18, wherein the step of moving the second core associated with the fixture to the deployed position comprises rotating the second core relative to the fixture.

20. The method of claim 18, further including the steps of:
attaching a pre-formed part to the object;
placing one or more removable cores into the object; and
placing one or more removable cores inside the injection mould prior to moulding a second portion of the object; and
over-moulding a membrane as part of the second portion of the object.

21-52. (canceled)
